SEQUENCE LISTING (8 sequences)

SEQ ID NO: 1 - DNA, 20 bp, Artificial Sequence
  Synthetic
ctgaacatcccgaggaactg

SEQ ID NO: 2 - DNA, 20 bp, Artificial Sequence
  Synthetic
ctctgagccactgcaaatga

SEQ ID NO: 3 - DNA, 22 bp, Artificial Sequence
  Synthetic
cttttgtgatcgtgacggctta

SEQ ID NO: 4 - DNA, 22 bp, Artificial Sequence
  Synthetic
tgatggtagggttttccgattc

SEQ ID NO: 5 - DNA, 21 bp, Artificial Sequence
  Synthetic
ggacaggactgaacgtcttgc

SEQ ID NO: 6 - DNA, 20 bp, Artificial Sequence
  Synthetic
atagccccccttgagcacac

SEQ ID NO: 7 - DNA, 20 bp, Artificial Sequence
  Synthetic
gctgctcataagactcggcc

SEQ ID NO: 8 - DNA, 20 bp, Artificial Sequence
  Synthetic
gtcacccaagtcccgtccta

(19) United States
(12) Patent Application Publication
(10) Pub. No.: US 2021/0270851 A1
(43) Pub. Date: Sep. 2, 2021

(54) BIOMARKER FOR DIAGNOSING AUTISM SPECTRUM DISORDER

(71) Applicant: The Board of Trustees of the Leland Stanford Junior University, Stanford, CA (US)

(72) Inventor: Karen J. Parker, Stanford, CA (US)

(21) Appl. No.: 16/975,114

(22) PCT Filed: Feb. 21, 2019
(86) PCT No.: PCT/US2019/019029

§ 371 Date: Aug. 21, 2020

Related U.S. Application Data
(60) Provisional application No. 62/634,142, filed on Feb. 22, 2018.

(51) Int. Cl. G01N 33/74; C12Q 1/6806; G01N 33/566

(52) U.S. Cl. CPC G01N 33/74; C12Q 1/6806; G01N 33/566; G01N 2496/00; G01N 2800/56; G01N 2800/38; G01N 2333/575

(57) ABSTRACT

The application is about a method for diagnosing autism spectrum disorder (ASD) in a human subject, comprising providing a device comprising a reagent for determining the concentration of arginine vasopressin (AVP) in a biological sample from the subject; and measuring the concentration of AVP in the sample using the device. Disclosed is also a method for diagnosing ASD, comprising providing a first device comprising a reagent for determining a concentration of AVP and a second device comprising a reagent for determining a concentration of one or more analytes selected from arginine vasopressin receptor 1a and oxytocin receptor, to determine the concentrations of AVP and of the one or more analytes. Disclosed is also a method of predicting severity of ASD in a male human subject, comprising providing a device for determining the concentration of AVP in cerebrospinal fluid, said device comprising a reagent for determining presence or absence of AVP; and measuring the concentration of AVP in a biological sample from the subject using the device. Disclosed is also a method of predicting likelihood of an ASD in a human subject, comprising providing a device for determining the concentration of AVP in cerebrospinal fluid, said device comprising a reagent for determining presence or absence of AVP; and measuring the concentration of AVP in cerebrospinal fluid using the device.

### CROSS-REFERENCE TO RELATED APPLICATIONS

This application is an International Patent Application, which claims benefit of U.S. Provisional Application Ser. No. 62/634,142, filed on Feb. 22, 2018.

### STATEMENT REGARDING GOVERNMENT INTEREST

This invention was made with Government support under contracts HD083629, MH100387 and R21HD079095 awarded by the National Institutes of Health. The Government has certain rights in the invention.

### TECHNICAL FIELD

The subject matter described herein relates to methods for diagnosing autism spectrum disorder and to methods for predicting and/or determining severity of autism spectrum disorder, in human subjects.

### BACKGROUND

Autism spectrum disorder (ASD) is a neurodevelopmental disorder characterized by deficits in social communication and interaction, as well as restricted, repetitive patterns of behavior, interests, or activities. ASD is clinically heterogeneous (e.g., cognitive capabilities range significantly) and ASD impacts an estimated 1 in 68 US children (Christensen et al., Morbidity and Mortality Weekly Report, Surveillance Summaries, 65(3); 1-23; 2016) with severe health, quality of life, and financial consequences for patients, families and/or society. ASD is currently diagnosed on the basis of behavioral criteria because its underlying disease mechanisms remain poorly understood. Consequently, there are no blood-based diagnostic tools to detect, or approved medications to treat, ASD's core features. Identification of robust biological substrates of disease status and symptomology in ASD patients is therefore urgently needed. There are currently no laboratory-based diagnostic tests by which to differentiate children with ASD.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting, in scope.

In one aspect, a method for diagnosing autism spectrum disorder (ASD) in a human subject is provided. The method comprises providing a device comprising a reagent for determining the concentration of arginine vasopressin (AVP) in a biological sample from the subject and measuring the concentration of AVP in the sample using the device. A diagnosis of ASD is affirmative, in one embodiment, when the AVP concentration is about 25-35% lower than an average AVP concentration in biological samples from a population of non-ASD human subjects.

In one embodiment, the biological sample is selected from the group consisting of cerebral spinal fluid (CSF), saliva, and urine.

In one embodiment, the device is an immunoassay comprising as the reagent an antibody for binding AVP.

In one embodiment, the device is an immunoassay comprising an antibody with specific binding to AVP.

In another embodiment, the device further comprises an antibody with a detectable label. In various embodiments, the detectable label is an enzyme, a radioactive isotope, or a fluorogenic molecule. In one embodiment, the device is a lateral flow immunoassay, an enzyme-linked immunoassay, or a radioimmunoassay.

In another embodiment, the device is a container comprising as the reagent a molecule for immunocapture of AVP and a nucleic acid probe linked to the molecule for immunocapture of AVP. Amplification of the probe and detection of its amplicons, if present, provide an approach for determining presence or absence of AVP in the sample. In one embodiment, the amplification of the probe is via polymerase chain reaction and, in another embodiment, the probe is amplified via isothermal amplification.

In another embodiment, the biological sample is CSF.

In another embodiment, a concentration of between about 0.1-20 pg/mL of AVP indicates an 80% chance or greater that a patient has ASD. In another embodiment, a concentration of AVP in the biological sample of less than 20 pg/mL is indicative of an 80% chance that the subject providing the sample has ASD.

In another embodiment, the concentration of equal to or less than about 20 pg/mL of AVP indicates an 80% chance that a patient has ASD.

In another embodiment, the concentration of between about 20-30 pg/mL or between about 24-26 pg/mL indicates that a patient is more than 50% likely to have ASD.

In another aspect, a method for diagnosing ASD in a human subject comprises providing a first device comprising a reagent for determining a concentration of AVP and a second device comprising a reagent for determining a concentration of one or more analytes selected from arginine vasopressin receptor 1a and oxytocin receptor; and contacting a biological sample from the human subject with the device, to determine the concentrations of AVP and of the one or more analytes, wherein a diagnosis of ASD is assigned to the subject if (i) the determined concentration of AVP is about 25-35% lower than a concentration of AVP in a population of non-ASD subjects and (ii) the determined concentration of the one or more analytes is about 20-30% lower than a concentration of AVP in a population of non-ASD subjects.

In one embodiment, the first device and the second device are provided in a kit comprised of the first and second devices.

In another embodiment, the biological sample is selected from the group consisting of cerebral spinal fluid, saliva, and urine.

In another embodiment, the concentration of AVP is determined in a cerebral spinal fluid sample and the concentration of one or more analytes is determined from a blood sample.

In another embodiment, the first device for determining the concentration of AVP is a container comprising as the reagent a molecule for immunocapture of AVP and a nucleic acid probe associated with the molecule for immunocapture of AVP. Amplification of the probe and detection of its amplicons, if present, provide an approach for determining quantitative or qualitative presence, or absence, of AVP in the sample.

In another embodiment, the first device for determining the concentration of AVP is an immunoassay comprising an antibody with specific binding to AVP.

In another embodiment, the second device is a container comprising as the reagent a primer set for amplification of arginine vasopressin receptor 1a or oxytocin receptor and a probe for detection of arginine vasopressin receptor 1a or oxytocin receptor amplicons.

In another embodiment, the second device is a container comprising as the reagent a primer set for amplification of arginine vasopressin receptor 1a or oxytocin receptor and a probe for detection of arginine vasopressin receptor 1a or oxytocin receptor amplicons.

In another aspect, a method of predicting severity of ASD in a male human subject is provided. The method comprises providing a device for determining the concentration of AVP in cerebrospinal fluid, the device comprising a reagent for determining presence or absence of AVP; and measuring the concentration of AVP in a biological sample from the subject using the device, wherein a concentration 50-60% lower than concentration in a subject without ASD is predictive of severe (e.g., 8 or higher on a scale of 10 as measured by the Autism Diagnostic Observation Schedule Calibrated Severity Score (ADOS-CSS)) ASD symptomology.

In another aspect, a method of predicting likelihood of an ASD in a human subject comprises providing a device for determining the concentration of AVP in cerebrospinal fluid, the device comprising a reagent for determining presence or absence of AVP; and measuring the concentration of AVP in cerebrospinal fluid using the device, wherein a concentration 25-35% lower than concentration in non-ASD subjects is predictive of ASD.

In addition to the exemplary aspects and embodiments described above, further aspects and embodiments will become apparent by reference to the drawings and by study of the following descriptions.

Additional embodiments of the present methods and the like will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

### BRIEF DESCRIPTION OF THE DRAWINGS

FIGS. 1A-1B are graphs showing the probability of being a low-social animal as a function of AVP concentration in cerebral spinal fluid (CSF; FIG. 1A) and of oxytocin (OXT) concentration in CSF (FIG. 1B). The data show that specific biological measures predict monkey social classification. The logistic regression model correctly classified 24 out of 27 monkeys (i.e., 89%). Low-social monkeys plotted above, and high-social monkeys plotted beneath, the dashed line in each graph are correctly classified. Each graph thus depicts a line that represents the model as a whole, and the effect of each biological measure is plotted corrected for the other variables in the analysis. Accordingly, the curves indicate directionality rather than magnitude of effect. P-values are reported to indicate the strength of the plotted relationships, and the biological measures are presented in order of their contribution to the predictive power of the model.

FIG. 2 is a graph of AVP concentration in CSF (FIG. 2A) for low-social and high-social animals. Data are presented as LSM ±SEM. CSF AVP concentration differed significantly between low-social and high-social monkey groups (N=27).

FIGS. 3A-3B demonstrate that CSF AVP concentration predicts group classification and differs between low-social and high-social monkeys in the replication cohort. FIG. 3A shows the probability of being a low-social animal as a function of mean AVP concentration (pg/mL) in cerebral spinal fluid, where the effect of CSF AVP concentration on predicted (line) and observed (circles) social group is plotted, corrected for the other variables in the analysis. Twenty-eight out of 30 monkeys (93%) were correctly classified. FIG. 3B shows the mean AVP concentration (pg/mL) in cerebral spinal fluid for low-social and high-social animals, where low-social monkeys are depicted with open circles and open bar, and high-social monkeys are depicted by closed circles and dotted bar.

FIGS. 4A-4B show that CSF AVP concentration predicts diagnostic status and differs between children with and without ASD. FIG. 4A shows the effect of CSF AVP on predicted (line) and observed (circles) diagnostic group, corrected for the other variables in the analysis. Children with ASD plotted above, and medical control (CON) children plotted beneath, the dashed line are correctly classified—as 13 out of 14 children (93%) were correctly classified. FIG. 4B shows the mean AVP concentration (pg/mL) in cerebral spinal fluid for low-social and high-social children, where children with ASD are depicted by open circles (FIG. 4A) and open bar (FIG. 4B), and medical control children are depicted by closed circles (FIG. 4A) and dotted bar (FIG. 4B).

FIG. 5 is a graph of probability of ASD diagnosis as a function of total neuropeptide receptor gene expression (the sum of the oxytocin receptor (OXTR)−ΔCT and the AVP receptor 1A (AVPR1A)−ΔCT) in children with (open circles) and without (closed circles) ASD, showing that total neuropeptide receptor gene expression predicts disease status in children with and without ASD.

FIGS. 6A-6D are bar graphs of blood neuropeptide measures total neuropeptide receptor gene expression (FIG. 6A), differential neuropeptide receptor gene expression (FIG. 6B), plasma AVP concentration (FIG. 6C) and plasma OXT concentration (FIG. 6D) for ASD and control subjects. Only total neuropeptide receptor gene expression differed significantly between the ASD and control groups.

FIGS. 7A-7C are graphs of social impairment measures SRS Total (Raw) Score (FIG. 7A) and RBS-R Stereotyped Behavior Subscale (FIG. 7B) and a cognitive measure, Stanford Binet IQ test (FIG. 7C), as a function of total neuropeptide receptor gene expression. The data shows that total neuropeptide receptor gene expression predicts symptom severity for core, but not associated features of ASD. FIGS. 7A-7B show that social impairments, as measured by the SRS Total (Raw) Score (FIG. 7A), Stereotypies, as measured by the RBS-R Stereotyped Behavior Subscale (FIG. 7B), are most severe in ASD children with the lowest levels of total neuropeptide receptor gene expression. FIG. 7C shows that cognitive ability, as measured by the Stanford Binet IQ test, is unrelated to total neuropeptide receptor gene expression.

FIGS. 8A-8B are bar graphs of CSF AVP concentration (pg/mL) and of CSF OXY concentration (pg/mL) for children with (open bars) and without (dotted bars) ASD.

FIG. 8C is a graph showing probability of ASD diagnosis as a function of CSF AVP concentration (pg/mL) in children with ASD (open symbols) and without ASD (closed sumbols).

FIGS. 8D-8F are graphs, respectively, of Autism Diagnostic Observation Schedule (ADOS) Calibrated Severity Score (CSS), ADOS social severity, and ADOS repetitive severity in male and female children with ASD, as a function of CSF AVP concentration (pg/mL), the data plotted as residuals from the least-squares line (i.e., both data and the regression line are corrected for other variables in the analysis). The significance of the interaction (i.e., the difference between the slope of the lines) is shown.

FIG. 9 provides a plot of AVP levels (standardized for age, sex and ethnicity) versus diagnosis status later in life.

FIG. 10 provides a plot of OXT levels (standardized for age, sex and ethnicity) versus diagnosis status later in life.

FIG. 11 provides a plot demonstrating that CSF AVP level (standardized for age, sex and ethnicity) predicts diagnosis (P<0.0001), while standardized OXT does not (P=0.6330).

FIG. 12 provides a bar graph demonstrating that individuals with an autism diagnosis later in life show lower CSF AVP levels prior to diagnosis (P=0.0007).

FIG. 13 provides a bar graph demonstrating that individuals with an autism diagnosis later in life do not differ in CSF OXT levels prior to diagnosis (P=0.6723).

### BRIEF DESCRIPTION OF THE SEQUENCES

SEQ ID NO.: 1 is a forward primer for OXTR.

SEQ ID NO.: 2 is a reverse primer for OXTR.

SEQ ID NO.: 3 is a forward primer for AVPR1A.

SEQ ID NO.: 4 is a reverse primer for AVPR1A.

SEQ ID NO.: 5 is a forward primer for HPRT1.

SEQ ID NO.: 6 is a reverse primer for HPRT1.

SEQ ID NO.: 7 is a forward primer for ubiquitin C.

SEQ ID NO.: 8 is a reverse primer for ubiquitin C.

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 pg/mL to 8 pg/mL is stated, it is intended that 2 pg/mL, 3 pg/mL, 4 pg/mL, 5 pg/mL, 6 pg/mL, and 7 pg/mL are also explicitly disclosed, as well as the range of values greater than or equal to 1 pg/mL and the range of values less than or equal to 8 pg/mL.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The compositions of the present disclosure can comprise, consist essentially of, or consist of, the components disclosed.

All percentages, parts and ratios are based upon the total weight of the topical compositions and all measurements made are at about 25° C., unless otherwise specified.

The phrase “pharmaceutically acceptable” is employed herein to refer to those compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in mammals (e.g., animals), and more particularly, in humans.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

### II. Methods of Diagnosis and of Predicting Severity of Autism Spectrum Disorder

In a first aspect, a method of diagnosing ASD in a human subject is provided. The method comprises determining the concentration of AVP in a biological sample from the subject using a device, as described infra. A diagnosis of ASD is affirmative, in one embodiment, when the AVP concentration is about 25-35% lower than an average AVP concentration in biological samples from a population of non-ASD human subjects. In another method, the concentration of arginine vasopressin receptor 1a and/or oxytocin receptor is determined in order to diagnose ASD and/or to assess severity of ASD. In this method, a diagnosis of ASD is assigned to the subject if (i) the determined concentration of AVP is about 25-35% lower than a concentration of AVP in a population of non-ASD subjects and (ii) the determined concentration of the one or more analytes is about 20-30% lower than a concentration of AVP in a population of non-ASD subjects.

The methods described herein may also be used to predict responsiveness to a particular biological or behavioral therapy for ASD. Clinical trials that have administered OXT to ASD patients have documented significant variability in responses to OXT treatment, and the present methods contemplate measuring neuropeptide concentration and neuropeptide receptor expression to predict treatment efficacy in subsequent neuropeptide trials.

Studies conducted in support of the methods will now be described with respect to Examples 1-4.

Example 1 describes a study where a primate model was used for ethological observations to identify naturally low-social male rhesus monkeys that also demonstrate differences in neuropeptide levels compared to socially competent, high-social monkeys. Using a discovery and replication design, CSF AVP was identified as a measure of group differences in monkey social functioning. These findings were replicated in an independent cohort, and it was confirmed in an additional monkey cohort that CSF AVP concentration is a stable trait-like measure. These findings were then translated to an ASD patient cohort and it was shown that CSF AVP concentration is lower in male children with ASD compared to medical control children, and that CSF AVP concentration predicts diagnostic status with high accuracy.

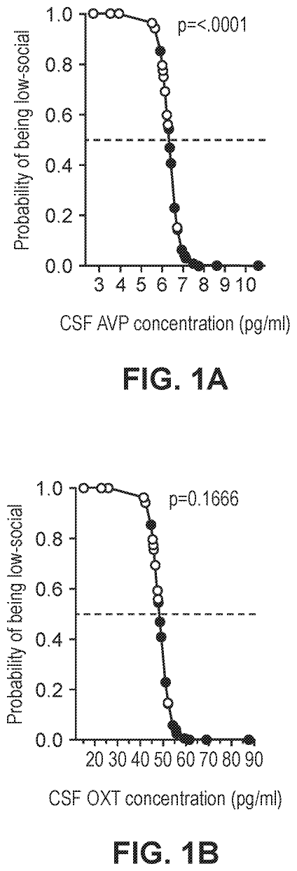

With continued reference to Example 1, biological signaling pathways (i.e., AVP, OXT) were measured in monkeys classified as low-social and high-social. It was found that CSF concentrations of AVP and OXT differed in the low-social and high-social animals, as shown in FIGS. 1A-1B. FIGS. 1A-1B show that CSF AVP concentration (FIG. 1A) predicted social classification, whereas CSF OXT concentration did not (FIG. 1B).

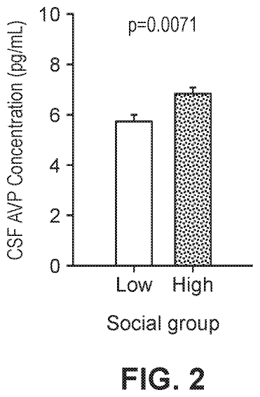

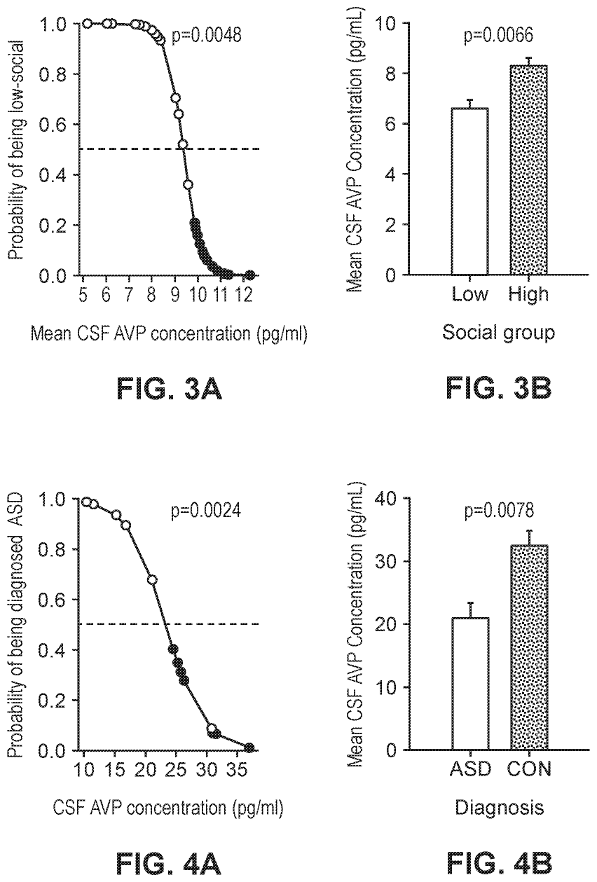

With continued reference to Example 1, tests were done to determine whether social classification predicted differences in CSF AVP biological measure. As seen in FIG. 2, CSF AVP concentrations were significantly lower in low-social vs. high-social monkeys. Having established that CSF AVP concentration was a measure of social classification in the discovery cohort, that the statistical winnowing strategy did not produce a false negative result, and that CSF AVP concentration was a stable trait-like measure in an additional cohort, a study was done to replicate this CSF AVP finding in an independent, replication cohort. This replication study is also described in Example 1, and in the data shown in FIGS. 3A-3B. As seen in FIGS. 3A-3B, CSF AVP concentration classified monkeys by group, with low-social monkeys showing lower CSF AVP concentrations compared to high-social monkeys.

To demonstrate that the data from the monkey model translates to humans, AVP concentrations in CSF samples that had been previously collected as part of routine medical care from seven male children with ASD and from seven male children without ASD (medical control children) between the ages of 6 to 12 years were quantified. It was found that CSF AVP concentration predicted diagnostic status, whereby individuals with lower CSF AVP concentrations were more likely to have been previously diagnosed with ASD, as shown in FIG. 4A. Like low-social monkeys, ASD patients showed significantly lower CSF AVP concentrations compared to control children, as shown in FIG. 4B.

Accordingly, in one embodiment, a method for diagnosing ASD or for assessing likelihood of a subject having an ASD is provided, by measuring AVP concentration in a biological sample, such as a CSF sample. An AVP concentration in the sample that is equal to or less than about 20 pg/mL indicates the subject is more than 80% likely to have ASD. In another embodiment, an AVP concentration in the sample that is equal to or less than about 20 pg/mL indicates an 80% or greater chance that a patient has ASD. In other embodiments, a concentration of AVP in the biological sample of between about 0.01-20 pg/mL, 0.1-20 pg/mL, 0.1-18 pg/mL, 0.1-15 pg/mL, 0.1-12 pg/mL, 0.5-20 pg/mL, 1-20 pg/mL, 1-18 pg/mL, 1-15 pg/mL, 1-12 pg/mL, or 1-10 pg/mL indicates an 80%, 85%, 90% or 95% chance or greater that the subject providing the sample has ASD. In another embodiment, an AVP concentration in the sample of between about 15-35 pg/mL, 15-30 pg/mL, 20-30 pg/mL, 22-28 pg/mL or 24-26 pg/mL indicates that a patient is more than 50% likely to have ASD.

Another study, detailed in Example 2, was designed to test in the same study population whether four blood-based neuropeptide measures (i.e., OXT and AVP peptide concentrations; OXTR and AVPR1A gene expression) correctly classified study participants as ASD or non-ASD (control). The study was also designed to evaluate whether these blood neuropeptide measures differed between children with ASD and control children, and to test whether the neuropeptide measures predicted symptom severity for core ASD features (i.e., social impairments and repetitive behaviors) but not associated features (i.e., intellectual impairment) in a well characterized child cohort.

In this study, 44 children with ASD (N=7 F, 37 M), and 24 unrelated neurotypical control children (N=6 F, 18 M) between the ages of 6 to 12 years participated. Demographic characteristics of the study subject are presented in Table 1 in Example 2, below. Blood samples were collected from the subjects for analysis of plasma AVP and OXT, and for quantification of oxytocin receptor (OXTR) and AVP receptor 1A (AVPR1A) gene expression, as described in Example 2. Ethnicity and blood collection time unexpectedly differed between children with and without ASD. To eliminate the possibility that these confounding effects could generate false positive or false negative results, a standard epidemiological approach to this problem was adopted, and these variables were included in the statistical models as blocking factors. IQ differed between groups, and the effect of IQ in the analyses was considered.

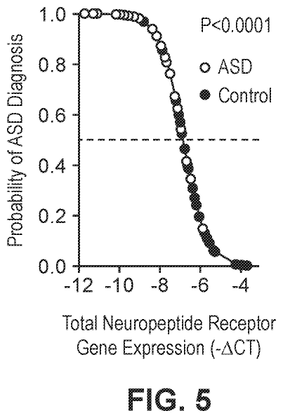

The logistic regression model correctly predicted disease status for 57 out of 68 (i.e., 84%) of the participants. As seen in FIG. 5, low levels of total neuropeptide receptor gene expression (i.e., sum of the OXTR and AVPR1A gene expression) predicted disease status in children with (open circles) and without (closed circles) ASD. Low plasma OXT concentration also predicted disease status. However, OXT concentration was significant in statistical models that included gene expression measures, indicating that OXT concentration serves as a moderator explaining additional variation, rather than being directly predictive. Differential neuropeptide receptor gene expression and plasma AVP concentration did not significantly predict disease status. In fact, a simple logistic regression, containing only total gene expression, no stratifying (blocking) factors, and no other biomarkers, still significantly predicted disease status, confirming that other biomarkers and stratifiers in model serve to explain additional noise around this central biological signal.

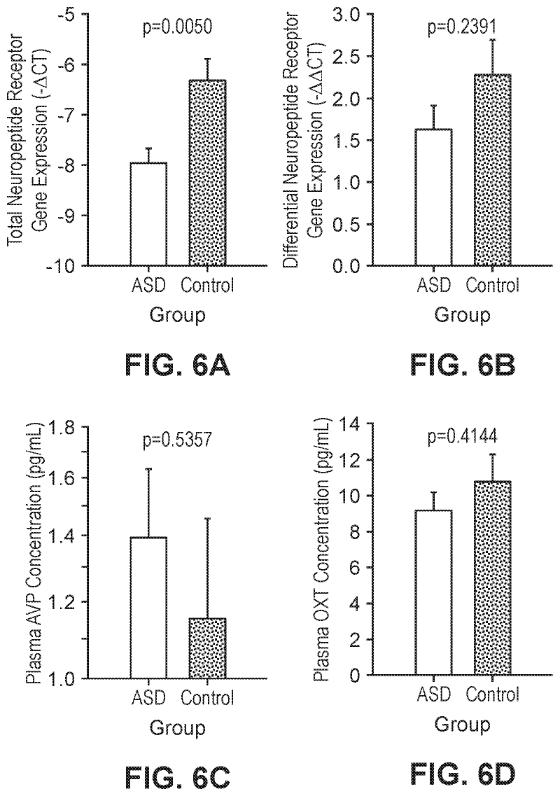

Total neuropeptide receptor gene expression was significantly lower in children with ASD, as seen in FIG. 6A. Differential neuropeptide receptor gene expression, shown in FIG. 6B, plasma AVP, shown in FIG. 6C, and plasma OXT concentrations, shown in FIG. 6D, did not differ significantly by disease status, strengthening the interpretation that OXT is a moderator of gene expression. Only total neuropeptide receptor gene expression differed significantly between the ASD and control groups.

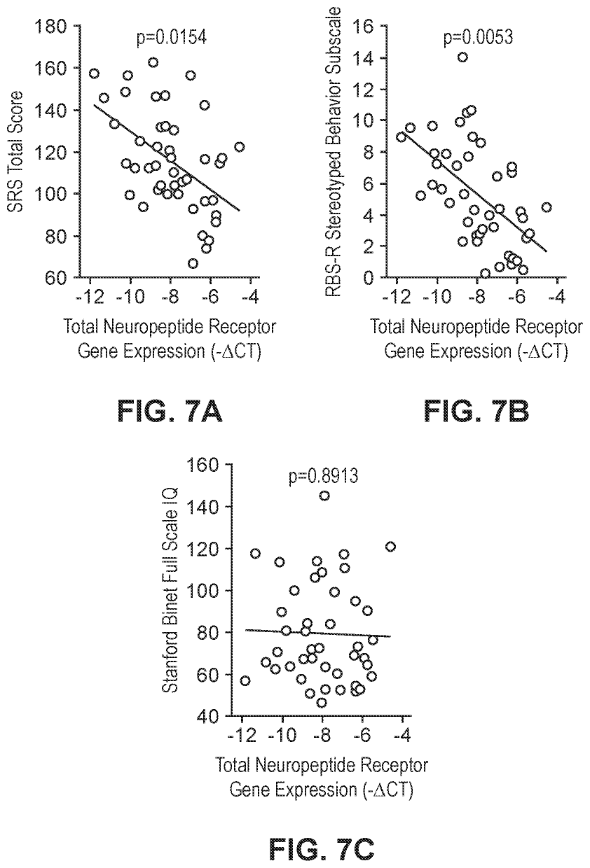

Data from this study also demonstrated that low levels of total neuropeptide receptor gene expression predicted greater social impairments as measured by the SRS Total (Raw) Score. This data is shown in FIG. 7A. No significant effect of the other neuropeptide measures on social functioning (P>0.05) was found. Low levels of total neuropeptide receptor gene expression also predicted greater severity of stereotypies as measured by the RBS-R Stereotyped Behavior Subscale, as seen in FIG. 7B. None of the other neuropeptide measures significantly predicted stereotyped behavior, nor were any significant results found in the other subscales for any neuropeptide measure. Also, neuropeptide receptor gene expression did not predict level of intellectual functioning as measured by IQ, as seen in FIG. 7C, thereby demonstrating more predictive specificity for core ASD features. None of the other neuropeptide measures predicted IQ either (P>0.05).

Another study, described in Example 3, was designed to test whether CSF neuropeptide (AVP and/or OXT) concentrations differ between ASD and control participants, and to test whether CSF neuropeptide concentrations correctly classify study participants as ASD vs. control. The study was also designed to test whether CSF neuropeptide concentrations predict symptom severity for core ASD features, particularly social impairments and to explore whether there is evidence for sex-specific ASD disease biology. A cohort of 72 human subjects was identified, composed of 48 males and 24 females. In the cohort, 36 subjects had ASD and 36 were non-ASD.

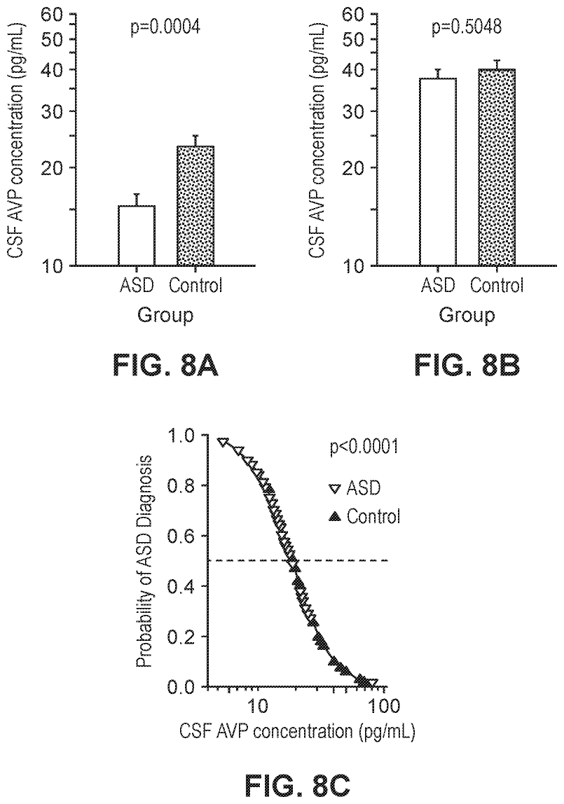

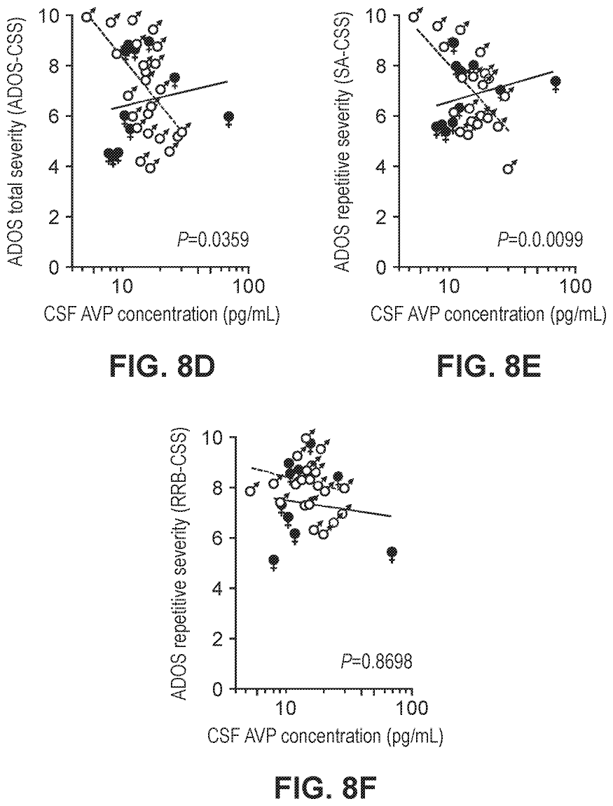

It was first tested whether children with ASD and control children differed in the CSF neuropeptide measures. As seen in FIG. 8A, CSF AVP concentration was significantly lower in the ASD compared to control group. No evidence for a CSF AVP concentration-by-sex interaction was found. Notable, as seen in FIG. 8B, CSF OXT concentration did not differ by group.

It was next studied whether CSF neuropeptide measures could accurately differentiate individual cases from controls. As seen in FIG. 8C, CSF AVP concentration significantly predicted ASD cases and non-ASD control subjects where 55 out of 72 (76%) individuals were correctly classified. Across the range of observed CSF AVP concentrations, the likelihood of ASD increased over 1000-fold, corresponding to nearly a 500-fold increase in risk with each 10-fold decrease in CSF AVP concentration. This relationship was observed in both males and females, as there was no evidence for a CSF AVP concentration-by-sex interaction. This effect was also specific to AVP, as CSF OXT concentration did not predict ASD likelihood in these same individuals.

Because CSF AVP concentration significantly predicted ASD likelihood, it was next evaluated whether low CSF AVP concentration predicted greater symptom severity in children with ASD, and whether these effects were specific to AVP (i.e., not apparent for CSF OXT as similarly evaluated). CSF AVP concentration significantly predicted overall symptom severity in a sex dependent manner, as seen in FIG. 8D, whereby lower CSF AVP concentration predicted greater symptom severity in males, but not in females (F1,27=0.2346; P=0.6320; β1,2±SE=0.9526±0.1.967), as measured by the Autism Diagnostic Observation Schedule Calibrated Severity Score (ADOS-CSS). Further investigation revealed that this effect on ADOS symptom severity was specific to the social domain (FIG. 8E), whereby low CSF AVP concentration predicted greater social impairments as measured by higher Social Affect (SA)-CSS in males, but not in females. In contrast, CSF AVP concentration did not predict severity scores for Restricted and Repetitive Behaviors (RRB)-CSS at all, as seen in FIG. 8F. No effect of CSF OXT concentration on any dimension of ADOS-CSS was found (P>0.05 for all tests).

Accordingly, based on the data and studies described herein, a method for diagnosis ASD and/or for predicting severity of ASD is contemplated. In the method, a device is used to measure the presence or absence of AVP in a biological fluid, such as saliva, urine or cerebral spinal fluid.

The device, in some embodiments, measures the amount of specific neuropeptides and/or expression of specific neuropeptide receptors in a biological sample and predicts the occurrence and severity of autism spectrum disorder (ASD). Neuropeptides include but are not limited to arginine vasopressin (AVP) and oxytocin (OXT), including isomers and metabolites thereof. As demonstrated in the Examples below that set forth primate and human data these neuropeptides and their receptors can be used to accurately predict the existence and severity of ASD.

In one embodiment of the method, a biological sample is taken from a patient, that sample is measured for neuropeptide concentration and/or neuropeptide receptor expression, and a determination is made as to whether the patient has ASD and/or the severity of the patients ASD symptoms based on the concentration of neuropeptide concentrations or neuropeptide receptor expression in the biological sample. In some embodiments that biological sample is taken from a patient's cerebral spinal fluid (CSF). In other embodiments, the biological sample is taken from a patient's blood, saliva or urine.

In some embodiments, the method can predict the occurrence or severity of ASD in a patient by measuring the concentration of a neuropeptide in a biological sample. In some embodiments, the neuropeptide is either AVP or OXT. In some embodiments, more than one neuropeptide may be evaluated for its ability to diagnose the occurrence or severity of ASD in a patient. In some embodiments, the biological sample may be taken from a patient's CSF. In other embodiments, the biological sample may be taken from a patient's blood, saliva or urine.

The presence of neuropeptide(s) in a biological sample may be achieved by known techniques in the art. Methods of determining neuropeptide concentrations are known in the art (Harlow and Lane, Antibodies: A Laboratory Manual New York: Cold Spring Harbor Laboratory (1988)). For example, in some embodiments, neuropeptide levels are quantified using an immunoassay. In some embodiments, neuropeptide levels may be quantified using a radioimmunoassay. In other embodiments, neuropeptide levels may be quantified using chromatography or spectroscopy, such as mass spectroscopy.

In some embodiments, the method comprises an enzyme-linked immunosorbent assay. In some embodiments, the enzyme-linked immunosorbent assay is selected from the group consisting of direct enzyme-linked immunosorbent assays, indirect enzyme-linked immunosorbent assays, direct sandwich enzyme-linked immunosorbent assays, indirect sandwich enzyme-linked immunosorbent assays, and competitive enzyme-linked immunosorbent assays. In alternative embodiments, the antibody used in the methods further comprises a conjugated enzyme, wherein the conjugated enzyme is selected from the group of enzymes consisting of horseradish peroxidases, alkaline phosphatases, ureases, glucoamylases, and β-galactosidases. In some embodiments, the enzyme-linked immunosorbent assay further comprises an alkaline phosphatase amplification system. In alternative embodiments, the methods further comprise at least one capture antibody, while in still further embodiments, the methods further comprise at least one detection antibody wherein the detection antibody is directed against the antibody directed against either OXT or AVP. In still further embodiments, the detection antibody further comprises at least one conjugated enzyme selected from the group consisting of horseradish peroxidase, alkaline phosphatase, urease, glucoamylase and β-galactosidase. In still further embodiments, the methods further comprise the step of quantitating the at least one neuropeptide in the biological sample.

In some embodiments, the neuropeptide expression levels are quantified using a quantitative polymerase chain reaction (qPCR), using primers for neuropeptides such as OXT and AVP, such as those described herein. Methods of quantifying neuropeptide expression are not limited by these examples. In another embodiment, methylation of neuropeptide genes is measured.

In some embodiments, the methods described herein are capable of predicting disease status in 70% of patients. In some embodiments, the methods are capable of predicting disease status in 80% of patients. In some embodiments, the methods are capable of predicting disease status in 90% of patients. In some embodiments, the methods are capable of predicting disease status in 95% of patients.

In some embodiments, a diagnosis of ASD is affirmative when the AVP concentration is at least about 25-35% lower than the concentration in a population of non-ASD subjects. In some embodiments, a diagnosis of ASD is affirmative when AVP concentration is at least about 30% lower than the concentration in a population of non-ASD subjects. In some embodiments, a diagnosis of ASD is affirmative when AVP concentration is at least about 30-40% lower than the concentration in a population of non-ASD subjects. In some embodiments, a diagnosis of ASD is affirmative when AVP concentration is at least about 20-30% lower than the concentration in a population of non-ASD subjects. In some embodiments, a diagnosis of ASD is affirmative when AVP concentration is at least about 20-60% lower than the concentration in a population of non-ASD subjects.

In some embodiments, the concentration of a single neuropeptide is used to predict disease status. For example, Example 3 demonstrates that CSF AVP concentrations significantly distinguish ASD patients from healthy controls. (Example 3, P<0.0001; FIG. 8). Across the range of observed CSF AVP concentration, the likelihood of a patient having ASD increases 1000-fold, corresponding to nearly a 500-fold increase in the risk with each 10-fold decrease in CSF AVP concentration. This effect is seen in both male and female patients.

In some embodiments, the neuropeptide may predict disease status, and in other embodiments the presence of other neuropeptides or neuropeptide receptors are included in the analysis. For example, as demonstrated in Example 2, low OXT concentration predicted disease status in statistical models that included gene expression measures for OXTR and AVPR1A.

Methods of predicting symptom severity in children with ASD is also contemplated. In some embodiments, CSF AVP concentrations significantly predict overall symptom severity. As demonstrated in Example 3, lower CSF AVP concentration predicts greater symptom severity in males with ASD. In some embodiments, symptom severity is measured by the Autism Diagnostic Observation Schedule Calibrated Severity Score (ADOS-CSS).

In some embodiments, neuropeptide levels correlate with a specific subtype of ASD symptoms. FIGS. 8A-8F demonstrated that low CSF AVP concentration predicted greater social impairments as measured by higher Social Affect (SA-CSS) in male subjects. In some embodiments, neuropeptide levels predict social impairment or social affect. In other embodiments, neuropeptide levels predict repetitive behaviors. Repetitive behaviors as defined by the Repetitive Behaviors Scale-Revised (RBS-R) includes six subscales of behavior (Stereotyped Behavior, Self-injurious Behavior, Compulsive Behavior, Ritualistic Behavior, Sameness Behavior and Restricted Behavior), for which psychometric validity is established. In some embodiments, neuropeptide levels can be used to identify the severity of individual subscales of repetitive behaviors.

In some embodiments, a concentration of AVP of 50-60% lower than the concentration in a subject without ASD is predictive of severe ASD symptomology (a score of 8 or higher the 10-point ADOS-CSS scale). In some embodiments, a concentration of 40-50% lower than the concentration in a subject without ASD is predictive of severe ASD symptomology. In some embodiments, a concentration of AVP of 55-65% lower than the concentration in a subject without ASD is predictive of severe ASD symptomology. In some embodiments, a concentration of AVP of 45-55% lower than the concentration in a subject without ASD is predictive of severe ASD symptomology.

In Example 3, symptom severity on a single day was measured and it was found that symptom severity correlated with AVP concentrations on that day. The results from Example 1 suggest that these neuropeptide measures are stable over time. Thus, in some embodiments, AVP concentrations in a biological sample will predict current symptom severity. In other embodiments, AVP concentrations in a biological sample will predict symptom severity over the course of several months, days and/or years following the measurement.

In some embodiments, expression of neuropeptide receptors is measured to diagnose an individual with ASD. In some embodiments, the receptors measured are the receptors for OXT and/or AVP. In some embodiments, the OXT receptor is (OXTR). In some embodiments, the AVP receptor is AVPR1A, AVPR1B or AVPR2. In some embodiments, both OXTR and AVPR1A are both measured to diagnose an individual with ASD.

In some embodiments, the neuropeptide receptor levels are quantified using a quantitative polymerase chain reaction (qPCR), using primer sequences for the OXTR and AVPR1A genes. Non-limiting examples of primers for OXTR and AVPR1A genes include:

OXTR forward
(SEQ ID NO.: 1)
5′-CTGAACATCCCGAGGAACTG-3′,
and
OXTR reverse
(SEQ ID NO.: 2)
5′-CTCTGAGCCACTGCAAATGA-3′;
AVPR1A forward
(SEQ ID NO.: 3)
5′-CTTTTGTGATCGTGACGGCTTA-3′,
and
AVPR1A reverse
(SEQ ID NO.: 4)
5′-TGATGGTAGGGTTTTCCGATTC-3′.

The relative expression of each gene is calculated based on the ΔCt value, where the results are normalized to the average Ct value of housekeeping genes, such as HPRT1 and UBC.

### Examples Housekeeping Genes Include:

HPRT1 forward 5′-GGACAGGACTGAACGTCTTGC-3′ (SEQ ID NO.: 5), and

HPRT1 reverse 5′-ATAGCCCCCCTTGAGCACAC-3′ (SEQ ID NO.: 6);

ubiquitin C (UBC) forward 5′-GCTGCTCATAAGACTCGGCC-3′ (SEQ ID NO.: 7), and

ubiquitin C (UBC) reverse 5′-GTCACCCAAGTCCCGTCCTA-3′(SEQ ID NO.: 8). Methods of quantifying neuropeptide receptor expression are not limited by these examples. In another embodiment, methylation of neuropeptide receptor genes is measured using known techniques in the art.

The expression of a neuropeptide receptor may either be measured alone or as part of a multidimensional neuropeptide expression analysis. In some embodiments, a multidimensional neuropeptide expression analysis is conducted to more powerfully diagnose disease status and symptom severity in children either with or without ASD. In one embodiment, gene expression of a vasopressin receptor is measured to identify patients as having or likely to have ASD and/or predict symptom severity, optionally in conjunction with measuring AVP concentration in a biological sample. In some embodiments, OXTR gene expression is measured to classify patients as having ASD and/or to predict symptom severity. In some embodiments, AVPR1A gene expression is measured to classify patients as having ASD and/or to predict symptom severity. In some embodiments, total combined expression of OTXR and AVPR1A is measured to classify patients as having ASD and/or to predict symptom severity. For example, as demonstrated in Example 2, OXTR and AVPR1A gene expression, when analyzed as part of such a multidimensional analysis were found to significantly predict disease status. Total neuropeptide receptor gene expression was significantly lower in children with ASD, as seen in FIG. 5A.

With reference again to Example 2, it was demonstrated that patients were correctly diagnosed with or without ASD. Various neuropeptide measures were used to correctly classify 84% of study participants as ASD or control. Accordingly, in some embodiments, a method for predicting disease status and/or for classifying disease status is provided, where the method provides accurate prediction and/or classification with a 70%, 80%, 90% or 95% confidence level.

Accordingly, a method for diagnosing ASD in a human subject is contemplated, where the method comprises providing a first device comprising a reagent for determining a concentration of AVP and a second device comprising a reagent for determining a concentration of one or more neuropeptide receptors selected from arginine vasopressin receptor 1a and oxytocin receptor; and contacting a biological sample from the human subject with the device, to determine the concentrations of AVP and of the one or more analytes. A diagnosis of ASD is assigned to the subject if (i) the determined concentration of AVP is about 25-35% lower than a concentration of AVP in a population of non-ASD subjects and (ii) the determined concentration of the one or more neuropeptide receptor is about 20-30% lower than a concentration of AVP in a population of non-ASD subjects. In some embodiments, the neuropeptide concentration may be at least 15-25% lower than a concentration of AVP in a population of non-ASD subjects. In some embodiments, the neuropeptide concentration may be at least 25-35% lower than a concentration of AVP in a population of non-ASD subjects.

In some embodiments, lower neuropeptide receptor expression levels predict greater symptom severity for ASD features selected from of social impairments and stereotyped behaviors. In some embodiments, lower neuropeptide receptor expression levels predict greater social impairment. In some embodiments, lower neuropeptide receptor expression levels predict more stereotyped behaviors in individuals with ASD.

In some embodiments, neuropeptide receptor expression correlates with a specific subtype of ASD symptoms. In some embodiments neuropeptide receptor expression predicts social impairment or social affect. In other embodiments neuropeptide receptor expression predicts repetitive behaviors. Repetitive behaviors as defined by the Repetitive Behaviors Scale-Revised (RBS-R) includes six subscales of behavior (Stereotyped Behavior, Self-injurious Behavior, Compulsive Behavior, Ritualistic Behavior, Sameness Behavior and Restricted Behavior), for which psychometric validity is established. In some embodiments, neuropeptide levels can be used to identify the severity of individual subscales of repetitive behaviors.

In some embodiments a multidimensional biomarker approach may be used to provide a diagnosis to a patient. Studies were performed to demonstrate a multidimensional approach to correctly diagnose a patient with ASD and/or quantify the severity of the patient's ASD symptoms.

In the study described in Example 2, neuropeptide receptor gene expression (OXTR and AVPR1A) were measured. Lower levels of total neuropeptide receptor gene expression predicted greater social impairment and stereotyped behavior in children with ASD, despite being unrelated to intellectual function. In subjects where the OXTR or AVP concentrations fail to diagnose ASD, inclusion of these peptide measures improved determining whether a subject has ASD. With regard to blood OXTR and AVP concentrations, these may serve as moderators explaining additional variation in, rather than being directly predictive of disease status.

Data can be managed using commercially available software. Logistical regression models implementing a Restricted Maximum Likelihood Generalized Linear Model (REML-GLIM) can be used to assess whether blood neuropeptide measures (i.e., OXT and AVP peptide concentrations, expression of OXTR and AVPR1A genes) predict disease status of patients with and without ASD. Age, time of blood collection, ethnicity, and sex can be included as control variables. When measuring expression of multiple neuropeptides or their receptors, the Principle Components Analysis may be used to yield orthogonal components for analysis.

A Least Squares General Linear Model (LS-GLM) can also be used to test whether neuropeptide measures differ between children with ASD and neurotypical controls. Each neuropeptide measure (total neuropeptide receptor gene expression, differential neuropeptide receptor gene expression, plasma neuropeptide concentration) can be tested in turn with the other neuropeptide measures as well as patient IQ, to ensure that any differences in for a given neuropeptide measure are not better explained by group differences in other neuropeptide measures or patient IQ. The assumptions of LS-GLM (homogeneity of variance, normality of error, and linearity) should be tested post hoc.

The LS-GLM can also be used to test whether the neuropeptide measures predict the core behavioral phenotypes in children with ASD. To assess social impairments the SRS Total Raw Score (instead of the sex-normalized T-score, which has lower resolution) or a similar test may be used. The same analysis may also be used on the SRS subscales whose psychometric validities are better established. The neuropeptide measures can also predict IQ (i.e., cognitive ability) to test for core vs. associated ASD feature specificity (thus, in this model, IQ was removed as a control variable). As before, the assumptions of LS-GLM are tested post-hoc.

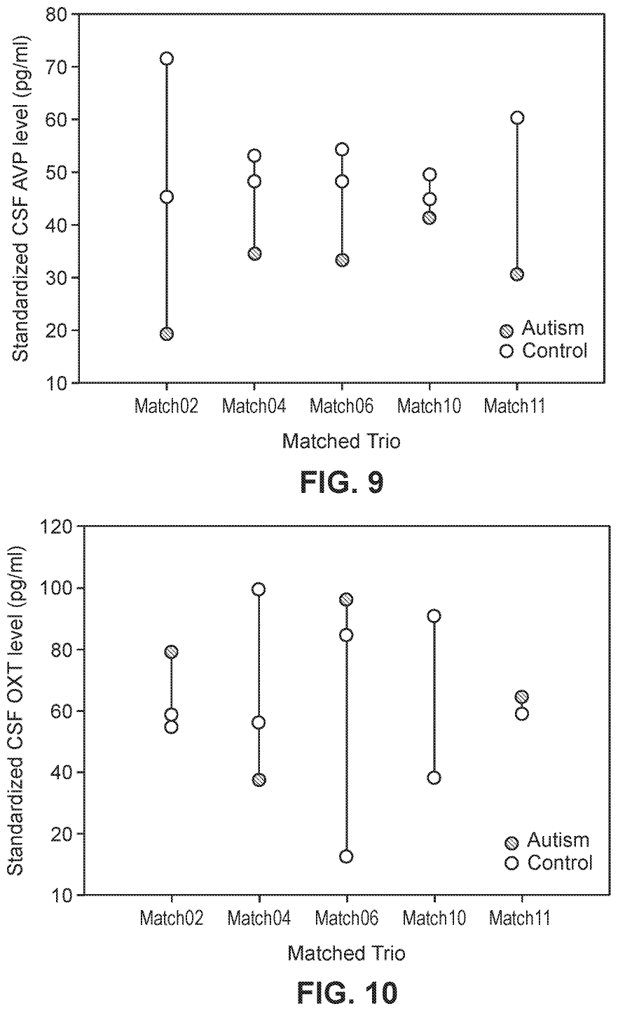

Example 4 provides description of a study undertaken to confirm the methods for diagnosis of the invention. Data from that study are provided in FIGS. 9-13.

The methods described for diagnosing ASD in a human subject comprise providing a device comprising a reagent for determining the concentration of a neuropeptide in a biological sample from the subject and measuring the concentration of the AVP in the sample using the device. Some embodiments of the method further comprise a second device comprising a reagent for determining a concentration of one or more neuropeptide receptors.

In some embodiments, said device comprises one or more capture reagents (such as, for example, at least one aptamer or antibody) for detecting one or more neuropeptides in a biological sample. In some embodiments, the device also contains a signal generating material. The device can also contain one or more reagents (e.g., solubilization buffers, detergents, washes, or buffers) for processing a biological sample. The device can also include, e.g., buffers, blocking agents, mass spectrometry matrix materials, antibody capture agents, positive control samples, and negative control samples.

In some embodiments, the device may include PCR primers for one or more neuropeptides or neuropeptide receptors. The device may also include PCR primers for one or more housekeeping genes. The device may also include a DNA array containing the complement of one or more neuropeptides or neuropeptide receptors, reagents, and/or enzymes for amplifying or isolating sample DNA. The device may also include reagents for real-time PCR, for example, TaqMan probes and/or primers, and enzymes.

In some embodiments, the device is compatible with other devices known in the art for reading protein or DNA/RNA concentrations, such as an ELISA microplate reader or a real-time PCR (or qPCR) thermocycler. In some embodiments, the device includes its own software and information such as protocols, guidance and reference data for diagnosing or evaluating the severity of ASD in a patient.

For example, a device can comprise reagents comprising at least capture reagent for quantifying one or more neuropeptides or neuropeptide receptor in a biological sample, and optionally (b) one or more algorithms or computer programs for performing the steps of comparing the amount of each neuropeptide or neuropeptide receptor quantified in the test sample to one or more predetermined cutoffs and assigning a score for each neuropeptide or neuropeptide receptor quantified based on said comparison, combining the assigned scores for each neuropeptide or neuropeptide receptor quantified to obtain a total score, comparing the total score with a predetermined score, and using said comparison to determine whether an individual has ASD. Alternatively, rather than one or more algorithms or computer programs, one or more instructions for manually performing the above steps by a human can be provided.

The methods described herein are contemplated for use with a human subject of any age. In some embodiments, the human subject is an infant. In other embodiments, the subject is an infant with a familial risk of ASD. In other embodiments, the subject to be diagnosed is a human child. In other embodiments, the subject is a human adult.

In one embodiment, the method is used for diagnosis of ASD in a patient that is under an age suitable for diagnosing ASD using behavioral methodologies, thus permitting therapeutic intervention in the patient prior to behavioral symptoms becoming apparent. In one embodiment, the patient is less than 5 years of age, less than 4 years of age, less than 3 years of age, less than 2 years of age or less than 1 year of age. In some embodiments, the method may diagnose ASD before behavioral symptoms have manifested in an individual, for example in an infant (e.g., 1 day to 24 months, 1 day to 18 months, 1 day to 12 months of age) with a familial risk of ASD. Such a diagnosis may lead to earlier behavioral or pharmacological intervention.

In some embodiments, the method is used to confirm a preliminary diagnosis made by traditional diagnosis based on behavioral data. In some embodiments, the patient has already received a preliminary diagnosis based on DSM-IV-TR (American Psychiatric Association, 2000) or DSM-5 criteria (American Psychiatric Association, 2013). In some embodiments, the patient has received a preliminary diagnosis based on the Autism Diagnostic Instrument-Revised (ADI-R) (Lord et al., J. Autism Dev. Disord. 24, 659-685 (1994)) and/or the Autism Diagnostic Observation Schedule, Second Edition (ADOS-2) (Lord, C., et a., 2012, Los Angel. CA West. Psychol. Corp.). In some embodiments the patient has already received a preliminary assessment of cognitive function using the Stanford Binet Scales of Intelligence, 5th Edition (Roid, G. H., 2003, Riverside Publishing Itasca, Ill.).

### III. Examples

The following examples are illustrative in nature and are in no way intended to be limiting.

### Example 1

### Arginine Vasopressin in Csf in Monkeys and Humans

42 male rhesus monkeys (selected from a pool of N=222 male monkeys) were identified that were expected to show extremes in social functioning (as detailed in the Materials and Methods section below). Ethological observations were performed on these individuals in their familiar social groups using focal-animal sampling methods, and a subset of N=15 low-social and N=15 high-social monkeys were identified, based on their social interactions with others in their troop. To test the validity of classifying animals from ethological data, personality trait ratings were also collected and a sociability score calculated. Sociability scores predicted the ethological classification of animals into low-social and high-social groups (LR ChiSq=19.94; P<0.0001).

Having validated the social groups, testing was done to determine whether low-social vs. high-social monkeys exhibited differences in biological signaling pathways (i.e., AVP, OXT, RAS-MAPK, PI3K-AKT). The measures included CSF concentrations of AVP and OXT; blood concentrations of AVP and OXT; blood OXTR and AVPR_{V1a} gene expression; and blood total and phosphorylated ERK, PTEN, and AKT. To eliminate false discovery a statistical winnowing strategy was used, whereby at each stage of analysis non-predictive and/or collinear biological measures were excluded from further consideration.

Initial testing was done to determine whether the biological dataset, considered as a whole, could accurately distinguish low-social from high-social monkeys. Discriminant analysis yielded a 93% correct social classification rate (LR ChiSq=26.36; p<0.0001). Logistic regression was used to identify which biological measures were significantly predictive. Including all of the biological measures yielded an over specified model, which is prone to false discovery. In the process of identifying a robust model, the following were excluded: blood concentrations of AVP and OXT, blood OXTR gene expression, and the phosphorylated-ERK/total-ERK ratio (all of which were non-predictive) and blood AVPR_{V1a} gene expression (which was collinear with other variables in the model). Thus, the stable logistic regression model included CSF concentrations of AVP and OXT as well as the ratios of phosphorylated-PTEN/total-PTEN and phosphorylated-AKT/total-AKT. Results are shown in FIGS. 1A-1D. Statistical analysis revealed that CSF AVP concentration (LR ChiSq=16.55; p<0.0001; FIG. 1A) and ratios of phosphorylated-PTEN/total-PTEN (LR ChiSq=6.792; p=0.0092; FIG. 1B) and phosphorylated-AKT/total-AKT (LR ChiSq=4.064; p=0.0438; FIG. 1C) in blood strongly and additively predicted social classification, whereas CSF OXT concentration did not (LR ChiSq=1.913; p=0.1666; FIG. 1D).

### CSF AVP Concentration Differs Between Low-Social and High-Social Monkeys and Positively Predicts Time Spent in Social Grooming.

Using a general linear model (GLM), tests were done to determine whether social classification predicted differences in significant biological measures independently. As seen in FIG. 2, CSF AVP concentrations were significantly lower in low-social vs. high-social monkeys (F_{1,18}=9.236; P=0.0071). For an initial validation of this result, it was tested whether CSF AVP concentration could predict a continuously distributed measure of social competence, and confirmed CSF AVP concentration positively predicted time spent in social grooming (F_{1,18}=7.2914; P=0.0146; partial r=+0.54).

### CSF AVP Concentration is a Predictor of Social Classification in a Replication Cohort.

Having established that: 1) CSF AVP concentration was a measure of social classification in the discovery cohort; 2) the statistical winnowing strategy did not produce a false negative result (data not shown); and 3) CSF AVP concentration was a stable trait-like measure in an additional cohort, a study was done to replicate this CSF AVP finding in an independent, replication cohort. N=164 male monkeys were observed, and social behavior observations were completed using a higher-throughput scan sampling-based method to identify a new cohort of N=15 low-social and N=15 high-social monkeys for CSF sample collection. As seen in FIGS. 3A-3B, CSF AVP concentration classified monkeys by group (LR ChiSq=7.969; p<0.0048), with low-social monkeys showing lower CSF AVP concentrations compared to high-social monkeys (F_{1,24}=8.847; P=0.0066).

CSF AVP Concentration Predicts Diagnostic Status and is Lower in Children with ASD.

To demonstrate that the data from the monkey model translates to humans, AVP concentrations in CSF samples that had been previously collected as part of routine medical care from N=14 male children (N=7 children with ASD; N=7 age-matched medical control children) were quantified. Using logistic regression, it was found that CSF AVP concentration predicted diagnostic status, whereby individuals with lower CSF AVP concentrations were more likely to have been previously diagnosed with ASD (LR-ChiSq=9.233; P=0.0024) as shown in FIG. 4A. Like low-social monkeys, ASD patients showed significantly lower CSF AVP concentrations compared to control children (F_{1,10}=11.02; P=0.0078), as shown in FIG. 4B. One ASD and control pair were sufficiently older than the other subjects in the analysis, so all analyses excluding this pair were rerun, and the results remained significant.

### Materials and Methods

Overall study design. Experimenters were blinded to monkey (i.e., low-social vs. high-social) and patient (i.e., ASD vs. control) groups during behavioral observations in monkeys and biological quantification (which included enzyme immunoassay, qPCR, and Western blot procedures) in both species.

Monkey Subjects

Subjects and study site. Subjects studied in the discovery and replication cohorts (i.e., cohorts 1 and 2, respectively) were N=206 male rhesus monkeys (Macaca mulatta), 1-5 years of age, born and reared at the California National Primate Research Center (CNPRC). Subjects lived in outdoor, half-acre (0.2 ha) field corrals, measuring 30.5 m wide×61 m deep×9 m high. Each corral contained up to 221 animals of all ages and both sexes. Subjects were tattooed as infants and dye-marked prior to behavioral observation for this study to facilitate easy identification. Monkeys had ad libitum access to Lixit-dispensed water, primate laboratory chow was provided twice daily, and fruit and vegetable supplements were provided twice weekly. Various toys, swinging perches, and other enrichments in each cage, along with outdoor and social housing, provided a stimulating environment. All procedures were approved by the relevant institutional IACUCs and complied with NIH policies on the care and use of animals.

### Subject Selection and Behavioral Data Collection for Monkey Cohort 1.

Subjects participated as infants in the colony-wide BioBehavioral Assessment (BBA) Program at CNPRC. The BBA comprises a set of highly standardized behavioral and physiological assessments focused on quantifying naturally occurring variation in temperament, behavioral responses, and pituitary-adrenal regulation as described elsewhere. BBA-enrolled monkeys were each tested between three to four months of age. To select subjects for cohort 1, an algorithm using an existing behavior dataset previously collected from N=80 adult male monkeys that were BBA “graduates” was developed. A factor scale (alpha=0.89) was created, and animals with z-scores of ≥−1.0 (low-social) or ≥+1.0 (high-social) were identified. Logistic regression revealed that BBA measures produced 88.9% classification. This statistical model was applied to a new set of BBA graduates (N=222), and selected N=42 of these monkeys for study in cohort 1.

Subjects were observed unobtrusively in their home field corrals. Inter-observer reliabilities of >85% agreement were established on behavioral categories, and age and sex classes of interaction partners prior to commencing experimental data collection. Each animal was then observed for two 10-min focal samples per day (0800-1030 and 1030-1300) over a 2-week period (called a “biweek”). A maximum of eight subjects, residing in one or two corrals, were observed per biweek. Behavior was recorded at 30-sec intervals using instantaneous sampling and time-ruled check-sheets. Five social behaviors were recorded: non-social (subject is not within an arm's reach of any other animal and is not engaged in play), proximity (subject is within arm's reach of another animal), contact (subject is touching another animal in a non-aggressive manner), groom (subject is engaged in a dyadic interaction with one animal inspecting the fur of the other animal using its hands or mouth), and play (subject is involved in chasing, wrestling, slapping, shoving, grabbing, or biting accompanied by a play face (wide eyes, open mouth without bared teeth) or a loose, exaggerated posture and gait; the behavior must be deemed non-aggressive to be scored). Only non-aggressive proximities and contacts were included for those two behaviors. Aggression was not analyzed here because data from a separate CNPRC cohort of n=78 comparably aged male monkeys had shown that animals of this age engage in aggression rarely (on average 0.29 aggressive events per hour), suggesting that aggression would have minimal impact on the data. The identities and age and sex classes of all social partners were recorded. At the end of a biweek, subjects were rated on 29 behavioral traits using a standardized instrument, with each trait evaluated on a 7-point scale.

Following completion of behavioral data collection, subjects were rank ordered on their total frequency of non-social behavior (summarized across the 320 focal behavior samples collected per subject). The N=15 monkeys with the greatest frequency of non-social behavior were classified as low-social, and the N=15 monkeys with the lowest frequency of non-social behavior (and therefore the highest frequency of all pro-social behaviors) were classified as high-social. Cerebrospinal fluid (CSF) and blood samples were subsequently collected from these N=30 subjects (see below).

### Subject Selection and Behavioral Data Collection for Monkey Cohort 2.

Data from cohort 1 demonstrated that the final social classification of the monkey subjects was essential. Thus, for the validation cohort more high-throughput behavioral methods were adopted, drawing the sample from all available male subjects that were born into, and were living in, the field corrals. Instead of focal sampling, a scan sampling approach was adopted to allow scoring multiple animals in the same group at the same time. Scan sampling, like the instantaneous sampling used for cohort 1, is a procedure that estimates durations of behavior (scan sampling is for groups, instantaneous sampling is for individuals). Thus, the same five core behaviors were estimated in both cohorts using an appropriate sampling technique to estimate behavioral durations. Prior to commencing experimental data collection, inter-observer reliabilities of >85% agreement were again established on behavioral categories, subject identities, and age and sex classes prior to commencing experimental data collection.

Subjects were observed unobtrusively in their home field corrals. Each observer conducted scan samples for a given corral during two observation periods per day (0900-1200 and 1300-1600 hr.). In each observation period, scan sampling was conducted at 20-minute intervals, at a rate of 18 scans per day, for a total of five days per corral. Thus, approximately N=90 scans were performed per corral. During each scan, the subjects in each corral were identified, and observers then recorded the occurrence of the following behaviors: non-social, proximity, contact, groom, and play as defined above. Also recorded were proximity and contact for aggressive episodes to confirm the aforementioned findings generated by an independent CNPRC research team that aggression in this age class was minimal. Consistent with these previous findings, no aggressive bouts were observed by the subjects during data collection for this cohort. Following completion of data collection, monkeys were rank ordered on their total frequency of non-social behavior (summarized across the 90 scan samples). The N=15 monkeys with the greatest frequency of non-social behavior were classified as low-social, and the N=15 monkeys with the lowest frequency of non-social behavior (and therefore the highest frequency of pro-social behavior) were classified as high-social. In order to further improve biological precision for cohort 2, two CSF samples from each subject were collected and the CSF AVP concentrations were averaged. Averaging the CSF AVP concentration also avoids collinearity between the two samples when predicting social group (see statistical analyses herein).

### Sample Collection and Processing Procedures.

Samples were collected between 0900-1100 to minimize any potential circadian effects on the biological measurements. Each subject was captured from his home corral, rapidly immobilized with telazol (5-8 mg/kg), and moved to an indoor procedure room. Supplementary ketamine (5-8 mg/kg) was used as needed to facilitate complete immobilization. Collection of both CSF and blood samples was accomplished within 10-15 min of initial cage entry; only one monkey per day was sampled from the same corral. The latency from cage entry to subject capture (to control for possible variation in stress effects on the biomarker measures) and collection time (to account for possible circadian effects on the biological measures) were recorded and used as statistical covariates.

Immediately following relocation, CSF (2 mL) was drawn from the cisterna magna using standard sterile procedure. CSF samples were immediately aliquoted into 1.5 mL siliconized polypropylene tubes and flash-frozen on dry ice. Next, whole blood samples (up to 25 mL) were drawn from the femoral vein and collected into: 1) EDTA-treated vacutainer tubes and placed on either wet ice (for neuropeptide quantification) or left at room temperature (for kinase quantification), and 2) PAXgene tubes and left at room temperature for 2 hours or longer (for neuropeptide receptor gene expression). Whole blood samples for neuropeptide quantification were promptly centrifuged (1600×g at 4° C. for 15 min), the plasma fraction aliquoted into 1.5 mL polypropylene tubes, and flash-frozen on dry ice. Whole blood samples for kinase signalling quantification were spun over a Ficoll-hypaque gradient and mononuclear cells collected from the interface were washed in PBS 2×, pelleted, and solubilized (in 50 mM Tris pH 7.4, 10 mM EGTA, 0.5% NP-40 and protease and phosphatase inhibitor cocktails). PAXgene tubes were subsequently transferred to −20° C. for 24 hours and then transferred to −80° C. per manufacturer's guidelines. All samples were stored at −80° C. until quantification. After sample collection, each subject was administered replacement fluids and ketoprofen as needed. Subjects were placed in a standard laboratory cage located in a hospital/transition room for recovery overnight, and then returned to their home corrals the next day.

### Neuropeptide Quantification.

CSF and blood OXT and AVP concentrations were quantified using commercially available enzyme immunoassay kits (Enzo Life Sciences, Farmingdale, N.Y.). These kits have been validated for use in rhesus monkeys and are highly specific and exclusively recognize OXT and AVP, respectively, and not related peptides (i.e., the OXT cross-reactivity with AVP is 0.6% and the minimum assay sensitivity is 11.7 pg/mL; and the AVP cross-reactivity with OXT is <0.001% and the minimum assay sensitivity is 3.39 pg/mL). A trained technician blinded to experimental conditions performed sample preparation and OXT and AVP quantification following established procedures recommended by the technical division of the assay manufacturer. Specifically, the CSF samples were directly assayed (without prior extraction) for OXT and AVP. The plasma samples were extracted for each hormone prior to assay to preclude known matrix interference effects of large blood borne proteins in the accurate quantification of the neuropeptides, using the following methods.

Plasma samples for use in OXT assays were extracted as follows: plasma samples (1000 μL/animal) were thawed in an ice bath, acidified with 0.1% trifluoroacetic acid (TFA), and centrifuged (17,000×g at 4° C. for 15 min). Phenomenex Strata-X columns (Phenomenex Inc., Torrance, Calif.) were activated with 4 mL of HPLC grade methanol followed by 4 mL of molecular biology grade water. Sample supernatants were applied and drawn through columns by vacuum following column activation, and eluted by sequentially applying 4 mL of wash buffer (89:10:1 water:acetonitrile:TFA) and 4 mL of elution buffer (20:80 water:acetonitrile).

Plasma samples for use in AVP assays were extracted as follows: Equal volumes of 40:60 butanol:diisopropyl ether were added to plasma samples (1000 μL/animal) prior to centrifugation at room temperature for 5 min at 8,000×g. The top organic layer was discarded and the aqueous solution transferred to a new mircocentrifuge tube. A 2:1 volume of ice cold acetone was then added to all samples prior to centrifugation at 4° C. for 20 min at 12,000×g. Supernatant was then transferred to 15 mL Falcon tubes and a volume of 5:1 ice cold petroleum ether was added. Samples were briefly vortexed, centrifuged at 1° C. for 10 min at 3350×g, and the top ether layer discarded.

Plasma samples for each neuropeptide assay were then evaporated at room temperature using compressed nitrogen. Each evaporated plasma sample was reconstituted in 250 μL of assay buffer prior to OXT and AVP quantification to provide sufficient sample volume to run each sample in duplicate wells (100 μL per well). Given the sensitivity limitations of the commercial assays, plasma extraction ensured that the plated samples contained high enough quantities of OXT or AVP to be read above the limit of detection. The program used to calculate pg/mL concentrations of OXT or AVP allows for extrapolation based on the sample concentration factor. That is, the program extrapolates the final OXT or AVP concentrations by dividing the results by the fold-difference in original sample volume. This method increases the concentration of OXT or AVP in each well, and ensures that each sample falls within the linear portion of the standard curve, above the assay's limit of detection, when it is initially read. All CSF and plasma samples were assayed in duplicate (100 μL per well) with a tunable microplate reader for 96-well format per manufacturer's instructions.

### Neuropeptide Receptor Quantification.

Measurement of OXTR and AVPR_{V1a} gene expression was done using protocols developed for rhesus monkeys. Total RNA was isolated and purified using a PAXgene blood RNA kit from blood stabilized in PAXgene RNA tubes (Qiagen, CA). The first strand cDNA synthesis reaction was carried out with iScript Reverse Transcription Supermix (Bio-Rad, CA) with a starting RNA quantity of 1 μg in a 20 μL final volume. qPCR was performed to determine OXTR and AVPR_{V1a} gene expression using RT² qPCR Primer Assays for Rhesus Macaque OXTR and AVPR_{V1a} (Qiagen, CA) and endogenous control (GAPDH, Life Technologies, CA) was used for normalization. qPCR was performed on the StepOnePlus Real-Time PCR System (Life Technologies, CA) with SYBR Green (Qiagen, CA). cDNA was PCR amplified in triplicate and Ct values from each sample were obtained using StepOnePlus software. Analyses were conducted using the comparative Ct method (2^{-ΔΔct}).

### Statistical Analyses.

Data were analysed using JMP Pro 13 (SAS Institute Inc., Cary, N.C.). Analysis of cohort 1 was done using a set of multidimensional biological measures, with an overall goal was to identify the biological measures most strongly associated with social classification. As discussed above, because false discovery is a risk in biomarker studies, a statistical winnowing strategy was adopted, whereby at each stage of analysis non-predictive or collinear biological measures were excluded from further consideration, via a series of increasingly demanding analyses. Significance for all statistical tests described below was determined to be P<0.05.

A quadratic (unequal covariance) discriminant analysis was used to test whether the biological measures considered as a whole could predict social classification. This technique is a form of directed machine learning that seeks to predict group as a linear combination of the predictors. Discriminant analysis answers the general question “can the biological measures predict social group?” but is agnostic as to which biological measures are drivers, and which may be mediators or moderators, of the social classification algorithm. CSF and blood concentrations of AVP and OXT, blood AVPRᵥ₁ₐ and OXTR gene expression, and ratios of phosphorylated-ERK/total-ERK, phosphorylated-PTEN/total-PTEN, and phosphorylated-AKT/total-AKT in blood were all included as predictors. The resulting confusion matrix (the table of actual versus predicted classifications) was then tested as logistic regression to yield the Likelihood-Ratio (and associated p-value) that the overall algorithm could predict social group given the biological measures. Two animals were excluded due to missing kinase data (i.e. N=28).

To test which biological measures were predictive, a logistic regression model was used. The model containing the full biological measurement panel showed over-specification and quasi-complete separation, indicating collinearity between predictors, and an artefactually over-precise classification prone to false positive results. Through a process of elimination of collinear variables, a final stable model was identified which maximized the number of biological measures included (i.e., CSF AVP, CSF OXT, phosphorylated-PTEN/total-PTEN, phosphorylated-AKT/total-AKT), as well as biologically relevant control variables (or ‘stratifiers’) (e.g., field corral, western blot). Two animals were excluded due to missing kinase data, and as a result, a third animal had to be excluded so that each field corral yielded at least one animal in each social group (i.e., N=27).

Whether any of the three key biological measures identified in the logistic regression showed group differences was tested. For CSF AVP concentration, a GLM was used where field corral, capture latency, and sample collection time were included as blocking factors, and social group was the predictor of interest. There was no need to control for assay run as all animals' samples were run on a single plate. N=30 monkeys yielded AVP data suitable for analysis. For the PTEN and AKT phosphorylation ratios, a GLM was used, again controlling for field corral, as well as western blot (to control for between-assay variance), and tested for the effect of social group. N=28 monkeys yielded kinase data suitable for analysis. Finally, to test whether CSF AVP concentration predicted time spent in social grooming (a measure recorded during behavioral data collection), the same GLM model was used and blocking factors as described above, with CSF AVP concentration included in the model as the predictor of interest (N=30). Time spent in social grooming was square-root transformed to meet the assumptions of GLM (normality of error, linearity and homogeneity of variance)

For the replication cohort (i.e., cohort 2), CSF AVP concentration was measured as this was the only biological measure that showed group differences in cohort 1. Thus, a logistic regression was used to test whether CSF AVP concentration could predict social group, controlling for capture latency and sample collection time, as well as behavioral observer. Testing was also done to measure whether social group conversely predicted CSF AVP concentration using a GLM that similarly controlled for capture latency, sample collection time, and behavioral observer. The same analyses as described above were used. All N=30 monkeys yielded biological data suitable for analysis. Average values were used for the two sampling time points for CSF AVP concentration, capture latency, and sample collection time for all cohort 2 analyses. As before, appropriate quality-control checks were performed for each analysis.

### Human Participants: Participants and Recruitment.

Participants were N=14 male children (N=7 boys with ASD and N=7 medical control boys) who were undergoing clinically indicated lumbar punctures and were recruited to participate in this research study. Participants were between 5 and 19 years old. Clinical indications for CSF collection for the study participants included rule-out diagnoses (e.g., clinical assessment to eliminate from consideration the possible presence of a condition or disease) and blood/tissue diseases such as leukemia that required CSF access in diagnosis or treatment. CSF aliquots for this study were either provided as an additional amount to the volume acquired for clinical purposes or reserved at the time of clinical procedure in lieu of disposal.

Inclusion criteria for all participants consisted of a clinically indicated reason for CSF collection, English speaking, any ethnicity, any gender, and between 6 months and 99 years of age. Children with ASD were required to meet diagnostic criteria for ASD (DSM-IV-TR or DSM-5) on the basis of clinical evaluation, and be free of other severe or co-morbid mental disorders (e.g., schizophrenia, bipolar disorder). Medical control children were required to be diagnosed with a medical problem other than ASD. Exclusion criteria for all children included declining to participate in the study or having parents who declined to participate in the study. Children with ASD (all of whom were male) were matched with control children 1:1 on the basis of gender and within a one-year band on age.

### CSF Sample Collection and Processing Procedures.

CSF was obtained using standard sterile procedures following administration of either local or general anesthetic. CSF was collected from the lumbar region by introduction of a 23-gauge spinal needle into the subarachnoid space at the L3-4 or L4-5 interspace below the conus medularis. After collection of the clinical CSF samples, CSF samples for research were immediately aliquoted into siliconized polypropylene tubes and flash-frozen on dry ice. All samples were stored at −80° C. until quantification.

### CSF Neuropeptide Quantification.

CSF AVP concentrations were quantified using the same commercially available enzyme immunoassay kits as used in the rhesus monkey experiments. (AVP is a highly conserved nonapeptide, and is structurally identical in rhesus monkeys and humans.) Sample preparation and AVP quantification were performed following established procedures. As with the monkeys, the human CSF samples were directly assayed (without prior extraction) and run in duplicate per the manufacturer's technical guidelines.

### Statistical Analyses.

Clinical data were managed using REDCap and analyzed using JMP Pro 13 (SAS Institute Inc., Cary, N.C.). All N=14 participants yielded biological data suitable for analysis. Patient data were analysed using a parallel approach employed in the monkey studies. Logistic regression was used to test whether CSF AVP concentration predicted diagnostic status. The first statistical model included CSF AVP concentration as a predictor, as well as standard control variables used in past clinical studies (i.e., age, sample collection time, ethnicity). There was no need to control for assay run as all patients' samples were run on a single plate. The initial model showed quasi-complete separation (i.e., particular combinations of predictors uniquely identified individuals, thereby bearing a high risk for false positives). Ethnicity was removed from the model as this factor was not significant (and there was no reason to a priori hypothesize that ethnicity would influence CSF AVP concentration). Age and sample collection time were retained as factors in the model to control for potential developmental changes and/or circadian variation in CSF AVP concentration. Next, like the monkeys, it was tested whether diagnostic status predicted CSF AVP concentration using a GLM, with the same control factors used in the stable logistic regression model. Significance for all statistical tests was determined to be P<0.05. The assumptions of GLM (normality of error, linearity and homogeneity of variance) were confirmed post-hoc for all analyses

### Monkey Cohort 1: Supplementary Quality Control Statistical Analyses and Results.

To confirm the specificity and validity of the statistical winnowing strategy, post-hoc quality control checks on monkey cohort 1's data were performed. First, testing for group differences in each of the biological measures was done. Second, since blood involves less invasive collection procedures than CSF, a separate analysis was performed to test whether blood AVP concentration predicted CSF AVP concentration. GLM was used with the same blocking factors as detailed herein in the main statistical analysis section. The assumptions of GLM (normality of error, linearity and homogeneity of variance) were confirmed post-hoc.

Consistent with the rationale behind the statistical winnowing strategy, no group differences were observed in any biological measures except CSF AVP concentration in cohort 1 (data not shown). Blood AVP concentration was also unrelated to CSF AVP concentration (F_{1,18}=0.001; P=0.9963). These quality control checks thus further supported a rationale for selecting CSF AVP concentration as the focus of all subsequent analyses.

### Monkey Cohort 3: Evaluating CSF AVP Concentration Stability Across Multiple Measurements.

To test whether CSF AVP concentration had trait-like qualities (i.e., if similar concentrations were evident across multiple samplings), and consistent with the 3R's (Replacement, Reduction, Refinement) principle that guides ethical animal research practice, CSF samples that had been previously collected and banked from a separate cohort of N=10 adult male monkeys, that ranged in age from 5-7 years were used. As with cohorts 1 and 2, cohort 3 subjects had been born and reared in the large outdoor corrals at the CNPRC. Cohort 3's CSF samples had been collected on four different occasions across a four-month period, with each collection separated by an average of 40 days (inter-collection interval range: 27 to 57 days). The same standard CSF collection procedures were employed as described above. As with cohorts 1 and 2, samples from monkey cohort 3 had also been collected in the morning, between 0800 and 1000. Finally, CSF AVP concentrations were quantified as described above using identical procedures.

These data were examined as a test-retest reliability estimate, for which a mixed model Intra-class Correlation Coefficient (ICC) was used. Following McGraw & Wong (Psychological Methods 1, 30-46 (1996)), a Case 3A ICC(C,1) i.e., an ICC of consistency estimated from a Restricted Maximum Likelihood Mixed Model, in which monkey is the subject, and time point is treated as a fixed effect repeated observation, was used, equivalent to the mean of all possible correlation coefficients between time points. To assess the significance of this result a repeated measures GLM was performed, and the significance of the random effect representing subject was tested. This analysis tests whether monkeys differed significantly from each other in a consistent manner from time point to time point.

CSF AVP concentration showed stability within-individuals across multiple time points (test retest reliability Intra-class Correlation Coefficient=0.78). This ICC was highly significant (F_{9,25}=12.88; P<0.0001) and considered a large effect size. Similar to the supplementary quality control analyses, this reliability analysis further supported a rationale for selecting CSF AVP concentration as the measure for ASD diagnosis.

### Example 2

### Diagnosis of ASD and of ASD Severity in Children with Autism

### Materials and Methods

### Participant Recruitment and Eligibility Criteria

Forty-four children with ASD (N=7 F, 37 M), and 24 unrelated neurotypical control children (N=6 F, 18 M) between the ages of 6 to 12 years participated. Participant demographic characteristics are presented in Table 1.

TABLE 1
Participant Characteristics

| | | Sex | | Ethnicity* | | | | | |
|---|---|---|---|---|---|---|---|---|---|
| Group | N | Female | Male | Caucasian | Asian | Other | Age (years) | Full-scale IQ* | Blood collection time (min)* |
| Autism | 44 | 7 | 37 | 12 | 12 | 20 | 8.54 ± 0.33 | 74.15 ± 3.98 | 14:04 PM ± 15.75 |
| Control | 24 | 6 | 18 | 16 | 3 | 5 | 8.71 ± 0.41 | 116.12 ± 2.57 | 12:32 PM ± 20.00 |

Fisher's exact test was used to test whether the distribution of individuals to different groups differed by sex and ethnicity. For age, full- scale IQ, and blood collection time, differences between groups were tested using a simple one-way general linear model (* = p < 0.05).
The values are reported as mean ± standard error,

Children with a diagnostic history of ASD underwent a comprehensive diagnostic evaluation to determine the accuracy of their previous diagnosis based on DSM-IV-TR (American Psychiatric Association, 2000) or DSM-5 criteria (American Psychiatric Association, 2013), which was confirmed with research diagnostic methods. These diagnostic methods included the Autism Diagnostic Instrument-Revised (ADI-R) (Lord et al., J. Autism Dev. Disord. 24, 659-685 (1994)) and/or the Autism Diagnostic Observation Schedule, Second Edition (ADOS-2) (Lord, C., et al., Autism Diagnostic Observation Schedule—2^{nd} Ed. 2012, Los Angeles, Calif. West. Psychol. Corp., 2012). The ADI-R and the ADOS-2 were administered by assessors trained by a research reliable clinician, and administration was reviewed for both initial and ongoing administration and coding reliability.

All participants were: 1) pre-pubertal; 2) in good medical health; and 3) willing to provide a blood sample. Participants with ASD were included if they had a Full-Scale IQ of 50 and above. Control participants were included if they had a Full-Scale IQ in or above the average range. Cognitive functioning was determined using the Stanford Binet Scales of Intelligence, 5th Edition (Roid, G. H., 2003, Riverside Publishing Itasca, Ill.). Exclusion criteria for children with ASD included: 1) a genetic etiology for ASD (e.g., Fragile X Syndrome); 2) a DSM-IV-TR or DSM-5 diagnosis of any severe mental disorder (e.g., schizophrenia, schizoaffective disorder, bipolar disorder), or 3) significant illness (e.g., serious liver, renal, or cardiac pathology). Participants taking medications were included as long as their medications were stable (i.e., for at least four weeks) before the blood draw. Control children were required to: 1) be free of neurological and psychiatric disorders in the present or past on the basis of medical history and 2) have no sibling diagnosed with ASD.

### Behavioral Phenotyping

The core behavioral features of ASD (i.e., social impairments and restricted, repetitive behaviors) were assessed using two instruments. 1) The SRS (Constantino et al., J. Autism Dev. Disord., 33, 427-433, 2003) is a norm-referenced questionnaire that measures social behavior in both clinical and non-clinical populations. The SRS Total Score is a sensitive measure (i.e., it strongly correlates with DSM criterion scores) with high reliability. 2) The Repetitive Behaviors Scale-Revised (RBS-R) (Lam and Aman, J. Autism Dev. Disord., 37, 855-866, 2007) assesses a wide range of restricted and repetitive behaviors. The RBS-R includes six subscales (Stereotyped Behavior, Self-injurious Behavior, Compulsive Behavior, Ritualistic Behavior, Sameness Behavior, and Restricted Behavior), for which the psychometric validity is established (Lam and Aman, J. Autism Dev. Disord., 37, 855-866, 2007).

### Blood Sample Collection and Processing Procedures

Twenty mL of whole blood was drawn from the child's antecubital region within two weeks of behavioral phenotyping. Blood samples were collected during daytime hours (i.e., between 10 AM and 5 PM) to reduce circadian effects on the biological measures of interest. Whole blood was collected into chilled EDTA-treated vacutainer tubes and immediately placed on wet ice. These samples were then promptly centrifuged (1600×g at 4° C. for 15 min), the plasma fraction aliquoted into polypropylene tubes, and flash-frozen on dry ice. Whole blood was also collected into PAXgene RNA tubes (Qiagen, CA) and processed per manufacturer's instructions. All samples were then stored at −80° C. until quantification.

### Quantification Procedures

OXT and AVP are primarily synthesized in the hypothalamus and released into systemic circulation by the posterior pituitary. The gold standard by which to measure these neuropeptide concentrations in blood is via immunoassay; such as enzyme-linked immunosorbent assays (ELISA). However, OXTR and AVPR1A are expressed in body tissues (Thibonnier, et al., Annu. Rev. Pharmacol. Toxicol., 41, 175-202 (2001)), including in blood lymphocytes (Yamaguchi, et al., Am. J. Physiol. Endocrinol. Metab., 287 E970-976, 2004). The gold standard for quantifying gene expression, qPCR, was used to assess blood mRNA levels of these neuropeptide receptors.

### Quantification of Plasma OXT and AVP Concentrations

Plasma OXT and AVP concentrations were quantified using commercially available enzyme immunoassay kits (Enzo Life Sciences, Inc., NY). These kits are highly specific and exclusively recognize OXT and AVP, respectively, and not related peptides (i.e., the OXT cross-reactivity with AVP is 0.6% and the AVP cross-reactivity with OXT is <0.001%). A technician blinded to experimental conditions performed sample preparation and OXT and AVP quantification following established procedures. Briefly, plasma samples (1000 μL/participant) for each peptide were extracted per manufacturer's instructions and evaporated using compressed nitrogen. Each evaporated sample was reconstituted in 250 μL of assay buffer prior to OXT and AVP quantification to provide sufficient sample volume to run each participant's sample in duplicate wells (100 μL/well). This practice ensured that the plated samples contained high enough peptide quantities to be read above the limit of detection (15 pg/mL for OXT and 2.84 pg/mL for AVP). Samples were assayed with a tunable microplate reader (Molecular Devices, CA) for 96-well format per manufacturer's instructions. Intra- and inter-assay coefficients of variation were below 10% for both analytes.

### Quantification of OXTR and AVPR1A Gene Expression Levels

Total RNA was isolated and purified using a PAXgene blood RNA kit from blood stabilized in PAXgene RNA tubes (Qiagen, CA). RNA integrity was assessed with the Agilent 2100 Bioanalyzer (Agilent Technologies, CA), and consistently found to have RIN values (RNA integrity numbers) greater than 9.5. The first strand cDNA synthesis reaction was carried out with QuantiTect reverse transcription kit (Qiagen, CA), with a starting RNA quantity of 1 μg in a 20 μl final volume. The primer sequence information for OXTR and AVPR1A genes was obtained from published studies and was designed as follows:

OXTR forward
(SEQ ID NO.: 1)
5′-CTGAACATCCCGAGGAACTG-3′,
and
OXTR reverse
(SEQ ID NO.: 2)
5′-CTCTGAGCCACTGCAAATGA-3′;
AVPR1A forward
(SEQ ID NO.: 3)
5′-CTTTTGTGATCGTGACGGCTTA-3′,
and
AVPR1A reverse
(SEQ ID NO.: 4)
5′-TGATGGTAGGGTTTTCCGATTC-3′.

Two housekeeping genes were selected for normalization using geNorm.

HPRT1 forward 5′-GGACAGGACTGAACGTCTTGC-3′ (SEQ ID NO.: 5), and

HPRT1 reverse 5′-ATAGCCCCCCTTGAGCACAC-3′ (SEQ ID NO.: 6)];

ubiquitin C (UBC) forward 5′-GCTGCTCATAAGACTCGGCC-3′ (SEQ ID NO.: 7), and

ubiquitin C (UBC) reverse 5′-GTCACCCAAGTCCCGTCCTA-3′(SEQ ID NO.: 8).

qPCR was performed on the StepOnePlus Real-Time PCR System (Life Technologies, CA) with SYBR Green (Thermo Fisher Scientific, MA). cDNA was PCR amplified in triplicate and Ct values from each sample were obtained using StepOnePlus software. The relative expression of each gene was calculated based on the ΔΔCt value, where the results were normalized to the average Ct value of HPRT1 and UBC.

### Statistical Analyses

Data were managed using REDCap and analyzed using JMP Pro 13 for Windows (SAS Institute Inc., NC). All analyses included N=44 ASD children, and (where appropriate) N=24 neurotypical control children. A logistic regression model, implemented as a Restricted Maximum Likelihood Generalized Linear Model (REML-GLIM), was used to assess whether blood neuropeptide measures (i.e., OXT and AVP peptide concentrations, expression of OXTR and AVPR1A genes) predict disease status of children with and without ASD. Age, time of blood collection, ethnicity, and sex were included as control variables (or ‘stratifiers’) in the initial model. This model showed over-specification and quasi-complete separation, indicating collinearity between predictors, and an artefactually over-precise classification prone to false positive results (Paul, D. A., Logistic regression using the SAS system: theory and application, SAS Inst. Corp USA, 1999). Since OXTR and AVPR1A gene expression was highly correlated, it was first considered using Principle Components Analysis (PCA) to yield orthogonal components for analysis. This neatly illustrated the collinearity of the gene expression measures, which loaded onto a single factor with loadings (correlation coefficients) of 0.8058 and 0.7049 for OXTR and AVPR1A, respectively. However, there were differences in the component structure when the ASD and control groups were processed separately. This precluded using a PCA to process the data from the two groups together. Given that OXT and AVP differ by only two amino acids, and their receptors likewise have a high degree of structural similarity, there is a substantial amount of documented crosstalk between these neuropeptides ligands at their receptors (Sala et al., Biol. Psychiatry, 69, 875-882 (2011); Schorscher-Petcu et al., J. Neurosci. 30, 8274-8284 (2010); Song et al., Psychoneuroendocrinology, 50, 14-19 (2014)). The total neuropeptide gene expression was calculated as the sum of the OXTR and AVPR1A gene expression to capture correlated expression of the two genes, and differential neuropeptide receptor gene expression as the difference between OXTR and AVPR1A gene expression to capture relative up or down regulation of these receptors. As plasma OXT and AVP concentrations were uncorrelated, they were included separately in the logistic regression model. The resulting model was robust, showing no evidence of over-specification or quasi-complete separation. Plasma AVP concentration was log-transformed in these and all other analyses to correct a skewed distribution. This confirmed the predictive power of total gene expression by running a single factor logistic regression (i.e., excluding all blocking factors and other biomarkers).

A Least Squares General Linear Model (LS-GLM), with the same control variables as those included in the logistic regression model, was used to test whether the neuropeptide measures differed between children with ASD and neurotypical controls. Each neuropeptide measure (total neuropeptide receptor gene expression, differential neuropeptide receptor gene expression, plasma AVP concentration, and plasma OXT concentration) was tested in turn, with the other three neuropeptide measures and IQ included in the model to ensure that any observed differences for a given neuropeptide measure were not better explained by group differences in the other neuropeptide measures or IQ. The assumptions of LS-GLM (homogeneity of variance, normality of error, and linearity) were tested post-hoc.

An LS-GLM with the same control variables as before was used to test whether the neuropeptide measures predicted core behavioral phenotypes in children with ASD. Each of the four neuropeptide measures and IQ were included in the model; as before, to exclude the possibility that a neuropeptide measure is significant merely due to IQ. This also allowed to test each neuropeptide measure in the context of the others in a single model. To assess social impairments, the SRS Total Raw Score (instead of the sex-normalized T-score, which has lower resolution) was used. Because the psychometric validity for the RBS-R Total Score is not well established, the same analyses on each RBS-R subscale was performed, but corrected to a critical p-value to 0.0083, to protect against multiple comparisons and to achieve the same family-level significance as the total score. It was also tested whether the neuropeptide measures predicted IQ (i.e., cognitive ability) to test for core vs. associated ASD feature specificity (thus, in this model, IQ was removed as a control variable). As before, the assumptions of LS-GLM were tested post-hoc.

### Results

### Participants

Participant demographic characteristics are presented in Table 1, above. Ethnicity and blood collection time unexpectedly differed between children with and without ASD. To eliminate the possibility that these confounding effects could generate false positive or false negative results, a standard epidemiological approach to this problem was adopted, and these variables were included in the statistical models as blocking factors. IQ differed between groups, and the effect of IQ in the analyses was considered.

### Biomarker Prediction of Disease Status

The logistic regression model correctly predicted disease status for 57 out of 68 (i.e., 84%) of the participants. Low levels of total neuropeptide receptor gene expression (i.e., sum of the OXTR and AVPR1A gene expression) predicted disease status (Likelihood Ratio Chi-square=17.16; P<0.0001; FIG. 5). Low plasma OXT concentration also predicted disease status (LR Chi-sq=4.700; P=0.0302). However, OXT concentration was significant in statistical models that included gene expression measures, indicating that OXT concentration serves as a moderator explaining additional variation, rather than being directly predictive. Differential neuropeptide receptor gene expression (LR Chi-sq=3.600; P=0.0578), and plasma AVP concentration (LR Chi-sq=0.1023; P=0.7491) did not significantly predict disease status. In fact, a simple logistic regression, containing only total gene expression, no stratifying (blocking) factors, and no other biomarkers, still significantly predicted disease status (LR Chi-sq=4.265; P=0.0389), confirming that other biomarkers and stratifiers in model serve to explain additional noise around this central biological signal.

### Total Neuropeptide Receptor Gene Expression Differs Between ASD and Control Children

Total neuropeptide receptor gene expression was significantly lower in children with ASD (F_{1,57}=8.5263; P=0.0050; FIG. 6A). Differential neuropeptide receptor gene expression (F_{1,57}=1.416; P=0.2391; FIG. 6B), plasma AVP (F_{1,57}=0.3883; P=0.5357; FIG. 6C), and plasma OXT concentrations (F_{1,57}=0.6760; P=0.4144; FIG. 6D) did not differ significantly by disease status, strengthening the interpretation that OXT is a moderator of gene expression.

### Total Neuropeptide Receptor Gene Expression Predicts Core, but not Associated, Features of ASD

Low levels of total neuropeptide receptor gene expression predicted greater social impairments as measured by the SRS Total (Raw) Score (F_{1,33}=6.533; P=0.0154; FIG. 7A). No significant effect of the other neuropeptide measures on social functioning was found (P>0.05). Low levels of total neuropeptide receptor gene expression also predicted greater severity of stereotypies as measured by the RBS-R Stereotyped Behavior Subscale (F_{1,33}=8.899; P=0.0053; FIG. 7B). None of the other neuropeptide measures significantly predicted stereotyped behavior, nor were any significant results found in the other subscales for any neuropeptide measure. Finally, neuropeptide receptor gene expression did not predict level of intellectual functioning as measured by IQ (F_{1,34}=0.0190; P=0.8913; FIG. 7C), thereby demonstrating more predictive specificity for core ASD features. Finally, none of the other neuropeptide measures predicted IQ either (P>0.05).

### Example 3

### CSF Vasopressin, Diagnostic Classification, and Social Symptom Severity in Children with Autism

A pediatric cohort diagnosed with DSM-IV-TR ASD was assembled for brain neuropeptide evaluation. Cases and controls were matched 1:1 on the basis of sex and within a 1-year band on age (combined cohort: N=72; N=48 males, N=24 females). The tables below set forth the participant characteristics.

| | | Sex | | Ethnicity | | | |
|---|---|---|---|---|---|---|---|
| | | Fe- | Male | Cau- | | Age | CSF collection |
| Group | N | male | | casian | Other | (years) | time (h:m:s) |
| Autism | 36 | 12 | 24 | 27 | 9 | 4.72 ± 0.27 | 10:55:08 AM ± |
| | | | | | | | 0:09:45 |
| Control | 36 | 12 | 24 | 19 | 17 | 4.66 ± 0.32 | 10:37:17 AM ± |
| | | | | | | | 0:23:33 |

Participant characteristics. Fisher's exact test was used to test whether the distribution of individuals to different groups differed by sex and ethnicity. For age, full-scale IQ, and blood collection time, differences between groups were tested using a simple one-way general linear model (* = p < 0.05). The values are reported as mean ± standard error.

| Behavioral Functioning | Mean | SD |
|---|---|---|
| Nonverbal Developmental Quotient* | 53.57 | 17.1 |
| Vineland-II Adaptive Behavior Composite Score | 64.28 | 8.64 |
| ADOS Symptom Severity Scores | Median | Range |
| Social Affect Calibrated Severity Score | 7 | (4, 10) |
| Restricted and Repetitive Behaviors | 9 | (5, 10) |
| Calibrated Severity Score | | |
| Total Calibrated Severity Score | 7 | (4, 10) |

It was first tested whether children with ASD and control children differed in the CSF neuropeptide measures. CSF AVP concentration was significantly lower in the ASD compared to control group (F1,66=14.20; P=0.0004; Regression coefficient, β1±SE=−0.09005±0.02390; i.e. ASD relative to control=66%, 95% CI=53-82%; FIG. 8A). No evidence for a CSF AVP concentration-by-sex interaction was found (F1,65=0.0521; P=0.8202). CSF OXT concentration did not differ by group (F1,66=0.4498; P=0.5048; FIG. 8B).

It was next tested whether CSF neuropeptide measures could accurately differentiate individual cases from controls. CSF AVP concentration significantly predicted cases and controls (Likelihood Ratio Chi-Square=15.14; P<0.0001; FIG. 8C). Across the range of observed CSF AVP concentrations, the likelihood of ASD increased over 1000-fold, corresponding to nearly a 500-fold increase in risk with each 10-fold decrease in CSF AVP concentration (Range Odds Ratio=1080; Unit Odds Ratio=494; β1±SE=−6.202±1.898). This relationship was observed in both males and females, as there was no evidence for a CSF AVP concentration-by-sex interaction (LR=0.7279; P=0.3936). This effect was also specific to AVP, as CSF OXT concentration did not predict ASD likelihood (LR Chi-Square: 0.5200; P=0.4710) in these same individuals.

Because CSF AVP concentration significantly predicted ASD likelihood, it was next tested whether low CSF AVP concentration predicted greater symptom severity in children with ASD, and whether these effects were specific to AVP (i.e., not apparent for CSF OXT as similarly evaluated). CSF AVP concentration significantly predicted overall symptom severity in a sex dependent manner (F1,27=4.878; P=0.0359; FIG. 8D), whereby lower CSF AVP concentration predicted greater symptom severity in males (F1,27=6.221; P=0.0190; β1,1±SE=−5.091±2.041), but not in females (F1,27=0.2346; P=0.6320; β1,2±SE=0.9526±0.1.967), as measured by the Autism Diagnostic Observation Schedule Calibrated Severity Score (ADOS-CSS). Further investigation revealed that this effect on ADOS symptom severity was specific to the social domain (F1,27=7.708; P=0.0099; FIG. 8E), whereby low CSF AVP concentration predicted greater social impairments as measured by higher Social Affect (SA)-CSS in males (F1,27=8.771; P=0.0063; β1,1±SE=−5.604±1.892), but not in females (F1,27=0.6229; P=0.4369; β1,2±SE=−1.439±1.823). In contrast, CSF AVP concentration did not predict severity scores for Restricted and Repetitive Behaviors (RRB)-CSS at all (F1,27=0.0274; P=0.8698; FIG. 8F). No effect of CSF OXT concentration on any dimension of ADOS-CSS was found (P>0.05 for all tests).

### Materials and Methods

### Participant Recruitment

Clinical indication for CSF collection included rule-out diagnoses (e.g., clinical assessment to eliminate from consideration the possible presence of a condition or disease) and blood/tissue diseases such as leukemia that required CSF access in diagnosis or treatment. CSF aliquots from these participants were either provided as an additional amount to the volume acquired for clinical purposes or reserved at the time of clinical procedure in lieu of disposal.

Inclusion criteria for all participants in the present study consisted of English speaking, between 1.5 and 9 years of age, and willingness to provide CSF for biological analysis (regardless of whether CSF was collected primarily for standard of care or research purposes). Children with autism were required to meet DSM-IV-TR criteria for Autistic Disorder which was confirmed with research diagnostic methods [(i.e., Autism Diagnostic Interview-Revised and Autism Diagnostic Observation Schedule (ADOS)]. Control children were required to be diagnosed with (or worked up for) a medical problem other than autism. All participants were required to be free of severe mental disorders. Exclusion criteria for all participants included having parents who declined to participate in the study.

### CSF Sample Collection and Processing Procedures

For participants with autism, CSF was collected under sedation following a 12-hour fasting period and preceded by fluid replacement. For control participants, CSF was collected under a variety of circumstances and involved either local or general anaesthetic. For all participants, CSF was obtained using standard sterile procedures by clinical staff. CSF was collected from the lumbar region by introduction of a 23-gauge spinal needle into the subarachnoid space at the L3-4 or L4-5 interspace below the conus medullaris. After sample collection CSF was immediately aliquoted into polypropylene tubes and flash-frozen on dry ice. All samples were stored at −80° C. until quantification.

### Participant Case-Control Matching

Children with autism who had sufficient available CSF sample volumes were matched with control children 1:1 on the basis of gender and within a one-year band on age. The final sample for neuropeptide quantification thus included N=36 children with autism (N=12 females, 24 males) and N=36 control children without autism (N=12 females, 24 males).

### CSF Neuropeptide Quantification

CSF arginine vasopressin (AVP) and oxytocin (OXT) concentrations were quantified using commercially available enzyme immunoassay kits (Enzo Life Sciences, Inc., Farmingdale, N.Y.). These kits are highly specific and exclusively recognize AVP and OXT, respectively, and not related peptides (i.e., the AVP cross-reactivity with OXT is <0.001%; the OXT cross-reactivity with AVP is <0.02%). A research team member blinded to experimental conditions performed sample preparation and neuropeptide quantification following established procedures recommended by the technical division of the assay manufacturer. Specifically, the CSF samples were directly assayed (without prior extraction) for AVP and OXT, and run in duplicate (100 μl per well) with a tunable microplate reader (Molecular Devices, CA) for 96-well format.

### Statistical Analyses

Data were managed using REDCap and analyzed using JMP Pro 13, and SAS 9.4 for Windows (SAS Institute Inc., Cary, N.C.). To test whether CSF AVP and/or OXT concentrations differed between children with and without autism, a Least-Squares General Linear Model (LS-GLM) was used. Participant age, time of CSF sample collection, ethnicity, and sex were included as control variables in the model. CSF AVP and OXT concentration were tested in turn. The interaction between sex and group were included in the initial models, and then removed when non-significant, following best practice. CSF AVP and OXT concentrations were log-transformed in these and all other analyses to correct a skewed distribution, and to meet the underlying assumptions of the analytical methods. The assumptions of LS-GLM (homogeneity of variance, normality of error, and linearity) were tested and confirmed post-hoc.

To test whether CSF AVP and/or OXT concentrations accurately differentiated autism cases from controls, a logistic regression model was used, implemented as a Restricted Maximum Likelihood Generalized Linear Model (REML-GLM). The same control variables (or ‘stratifiers’) as those included in GLM model were used. Initially, interactions between each of the neuropeptide measures and sex were included in the model to test whether CSF AVP and/or OXT concentrations were sex-specific in predicting group. These interactions were not significant and were removed from the final analyses, following best practice for linear model design. The resulting model was robust, showing no evidence of over-specification or quasi-complete separation.

To test whether CSF AVP and/or OXT concentrations predicted symptom severity in the autism group, a LS-GLM was used, with the same control variables as before. One participant did not have available ADOS data, thus, N=35 for this analysis. Both neuropeptide measures, and their interactions with sex, were included in the initial model. To minimize the risk of false discovery, overall symptom severity was examined using the ADOS-Calibrated Severity Score (CSS). This analysis showed a significant interaction of CSF AVP concentration and sex, but not of CSF OXT concentration and sex (the latter of which was subsequently removed from the model). The same model was then used to test whether CSF AVP concentration, CSF OXT concentration, and the interaction of CSF AVP concentration and sex predicted symptom severity specifically on the Social Affect-CSS and the Restricted and Repetitive Behaviors-CSS. These secondary analyses were corrected for false discovery by setting a critical alpha=0.025 to account for multiple testing within the ADOS instrument. The assumptions of LS-GLM were tested post-hoc, and no transformations of the severity scores were required.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

### Example 4

### Screen for Data Integrity

A study was undertaken to confirm the methods of the invention. Subjects were drawn from a historical sample of 11 age, gender, and ethnicity matched trios with one Autism case, and two controls per trio. Trios were excluded from the primary analysis if the autism case presented with unrelated psychiatric comorbidities (these matched trios were included in follow up exploratory analyses). Trios were excluded if there was not viable biological data from at least one autism case and one control.

The initial data set of 11 matched-trios (1 case, 2 controls each) was identified, which were matched on age, ethnicity and gender. Trios showing complex diagnoses were excluded (i.e. ASD+secondary non-ASD related Dx), and associated controls (i.e. 4/11 trios), leaving seven trios. Initial analyses show that complex Dx are a distinct group c.f “simple” ASD diagnosis. Further trios were excluded where case (proband) has no viable biological data (2), leaving five trios in the study.

A logistic regression was first used to ask if CSF biomarkers could predict later diagnosis. Given the small number of trios, stratifying by trio lead to an over-specified model. Alternatively, the biomarker data was standardized by subtracting the mean value for each trio (which effectively controls for age, ethnicity, and gender). AVP perfectly predicted five of five (5/5) Autism cases, and nine of nine (9/9) controls (Likelihood Ratio Chi-sq=18.25; P<0.0001). In contrast, OXT did not show this correlation (LR Chi-sq=0.2279; P=0.6330). Similar results were seen when both biomarkers were included in the same logistic regression model.

That initial analysis was followed by a simple General Linear Model to ask whether neuropeptide biomarker levels differed between cases and controls. Accordingly, Autism cases showed significantly lower AVP levels than controls (F1,12=20.28; P=0.0007). In contrast, no significant difference was seen for OXT levels (F1,11=0.1898; P=0.6723).

In exploratory follow-up analyses, trios were included where the autism case presented with other comorbidities. The analysis revealed that there was some evidence that these individuals might show a more complex biomarker profile (namely elevated AVP over controls and simple cases, and lower OXT levels versus controls). Logistic regression was used to successfully distinguish these individuals from controls and secondarily, the simpler autism cases (i.e., it was possible to predict individuals who would develop autism in general, and individuals at risk for secondary comorbidities).

FIGS. 9-13 provide data obtained from the analyses described in this Example.

FIG. 9 provides a plot of AVP levels (standardized for age, sex and ethnicity) versus diagnosis status later in life.

FIG. 10 provides a plot of OXT levels (standardized for age, sex and ethnicity) versus diagnosis status later in life.

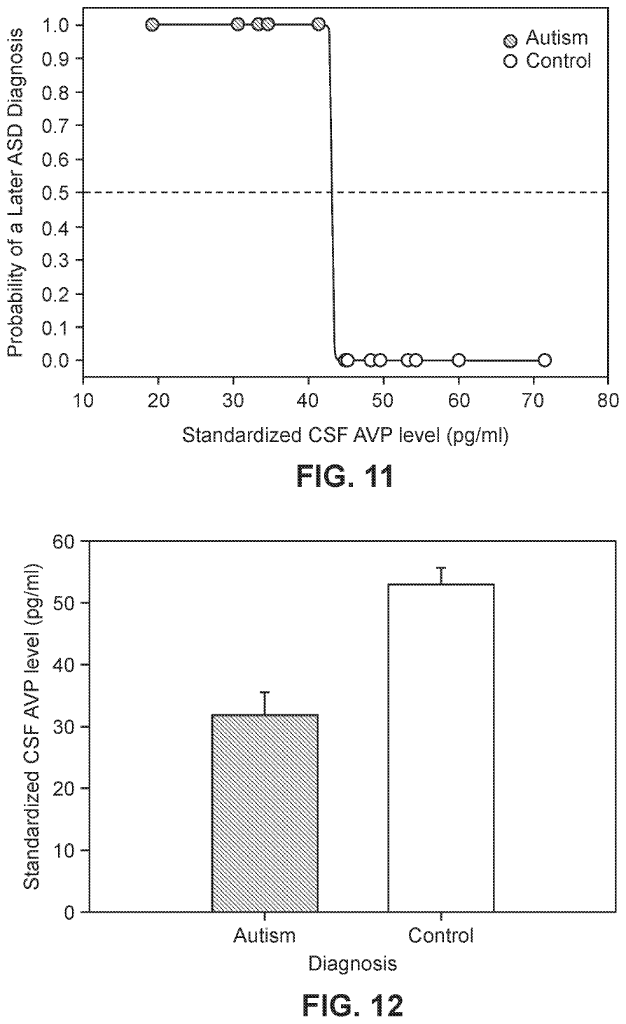

FIG. 11 provides a plot demonstrating that CSF AVP level (standardized for age, sex and ethnicity) predicts diagnosis (P<0.0001), while standardized OXT does not (P=0.6330).

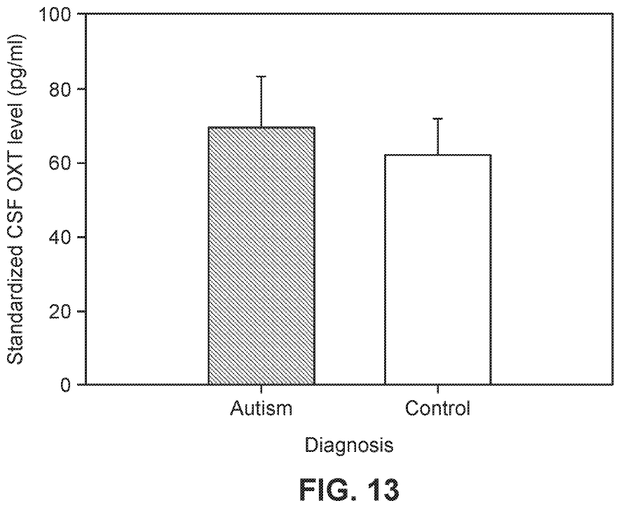

FIG. 12 provides a bar graph demonstrating that individuals with an autism diagnosis later in life show lower CSF AVP levels prior to diagnosis (P=0.0007).

FIG. 13 provides a bar graph demonstrating that individuals with an autism diagnosis later in life do not differ in CSF OXT levels prior to diagnosis (P=0.6723).

1. A method for diagnosing autism spectrum disorder (ASD) in a human subject, comprising:
providing a device comprising a reagent for determining the concentration of arginine vasopressin (AVP) in a biological sample from the subject; and
measuring the concentration of AVP in the sample using the device, wherein a diagnosis of ASD is affirmative when the AVP concentration is about 25-35% lower than an average concentration of AVP in a population of non-ASD subjects.

2. The method of claim 1, wherein the biological sample is selected from the group consisting of cerebral spinal fluid, saliva and urine.

3. The method of claim 1, wherein the device is a container comprising as the reagent an antibody for binding to AVP, the antibody associated with a nucleic acid probe.

4. The method of claim 3, wherein the device further comprises a primer set for amplification by polymerase chain reaction or by isothermal amplification of the probe.

5. The method of claim 1, wherein the device is an immunoassay comprising an antibody with specific binding to AVP.

6. The method of claim 5, wherein the device further comprises an antibody with a detectable label.

7. The method of claim 6, wherein the detectable label is an enzyme, a radioactive isotope, or a fluorogenic molecule.

8. The method of claim 1, wherein the biological sample is cerebral spinal fluid.

9. The method of claim 8, wherein a concentration of 0.1-20 pg/mL of AVP indicates an 80% or greater chance that a patient has ASD.

10. The method of claim 8, wherein in a concentration of less than about 20 pg/mL of AVP indicates an 80% chance or greater that a patient has ASD.

11. The method of claim 8, wherein a concentration of between about 20-30 pg/mL indicates that a patient is more than 50% likely to have ASD.

12. A method for diagnosing ASD in a human subject, comprising:
providing a first device comprising a reagent for determining a concentration of AVP and a second device comprising a reagent for determining a concentration of one or more analytes selected from arginine vasopressin receptor 1a and oxytocin receptor; and
contacting a biological sample from the human subject with the first device and the second device, to determine the concentrations of AVP and of the one or more analytes, wherein a diagnosis of ASD is assigned to the subject if (i) the determined concentration of AVP is about 25-35% lower than a concentration of AVP in a population of non-ASD subjects and (ii) the determined concentration of the one or more analytes is about 20-30% lower than an average concentration of AVP in a population of non-ASD subjects.

13. The method of claim 12, wherein the first device and the second device are provided in a kit comprise of the first and second devices.

14. The method of claim 12, wherein the biological sample is selected from the group consisting of cerebral spinal fluid, saliva and urine.

15. The method of claim 12, wherein the concentration of AVP is determined in a cerebral spinal fluid sample and the concentration of one or more analytes is determined from a blood sample.

16. The method of claim 12, wherein the first device for determining the concentration of AVP is a container comprising as the reagent an antibody for binding to AVP, the antibody associated with a nucleic acid probe.

17. The method of claim 12, wherein the first device for determining the concentration of AVP is an immunoassay comprising an antibody with specific binding to AVP.

18. The method of claim 12, wherein the second device is a container comprising as the reagent a primer set for amplification of arginine vasopressin receptor 1a or oxytocin receptor and a probe for detection of arginine vasopressin receptor 1a or oxytocin receptor amplicons.

19. A method of predicting severity of ASD in a male human subject, comprising:
providing a device for determining the concentration of AVP in cerebrospinal fluid, said device comprising a reagent for determining presence or absence of AVP; and
measuring the concentration of AVP in a biological sample from the subject using the device, wherein a concentration 50-60% lower than concentration in a subject without ASD is predictive of severe (8 or higher on a scale of 10) ASD symptomology.

20. (canceled)
